(19) United States
(12) Patent Application Publication
(10) Pub. No.: US 2026/0132403 A2
(43) Pub. Date: May 14, 2026

(54) ANTISENSE OLIGONUCLEOTIDES FOR TARGETING PROGRANULIN

(71) Applicants: Jesper WORM, Hørsholm (DK); Lars JOENSON, Hørsholm (DK); Mads MANSØ, Hørsholm (DK); Dorthe VANG, Hørsholm (DK)

(72) Inventors: Jesper WORM, Copenhagen (DK); Lars JOENSON, Viby Sjaelland (DK); Mads MANSØ, Hvidovre (DK); Dorthe VANG, Hvidovre (DK)

(73) Assignee: Hoffmann-La Roche Inc., Little Falls, NJ (US)

(21) Appl. No.: 18/982,750

(22) Filed: Dec. 16, 2024

Related U.S. Application Data
(63) Continuation of application No. PCT/EP2023/066081, filed on Jun. 15, 2023.

(30) Foreign Application Priority Data
Jun. 17, 2022 (EP) 22179662.6

(51) Int. Cl. C12N 15/113

(52) U.S. Cl. CPC C12N 15/113; C12N 2310/11; C12N 2310/314; C12N 2310/315; C12N 2310/321; C12N 2310/3341

(57) ABSTRACT

Antisense oligonucleotides for altering the splicing pattern of progranulin, and their use in the treatment of neurological disorders. The antisense oligonucleotides are modified to better increase up-regulation or expression restoration of the Exon1-Exon2 progranulin splice variant in cells.

### SEQUENCE LISTING

The instant application contains a Sequence Listing which has been submitted electronically in XML format and is hereby incorporated by reference in its entirety. Said XML copy, created on Feb. 28, 2025, is named 51551-025001_Sequence_Listing_2_28_25.xml and is 162,348 bytes in size.

### FIELD OF INVENTION

The present invention relates to antisense oligonucleotides which alter the splicing pattern of progranulin, and their use in the treatment of neurological disorders. Such antisense oligonucleotides may up-regulate or restore expression of the Exon1-Exon2 progranulin splice variant in cells.

### BACKGROUND

Progranulin (PGRN) is a highly conserved secreted protein that is expressed in multiple cell types, both in the CNS and in peripheral tissues.

Deficiency of the secreted protein progranulin in the central nervous system causes the neurodegenerative disease frontotemporal dementia (FTD). Pathogenic progranulin mutations lead to a loss of about 50% in progranulin levels through haploinsufficiency and to intraneuronal aggregation of TDP-43 protein. Progranulin plays a supportive and protective role in numerous processes within the brain, including neurite outgrowth, synapse biology, response to exogenous stressors, lysosomal function, neuroinflammation, and angiogenesis in both cell autonomous and non-autonomous manners.

Both directly and via its conversion to granulins, progranulin regulates lysosomal function, cell growth, survival, repair, and inflammation. Progranulin has a major role in regulation of lysosomal function associated microglial responses in the CNS. Autosomal dominant mutations of the progranulin gene leading to protein haploinsufficiency are linked to familial frontotemporal dementia with neuropathologic frontotemporal lobar degeneration (FTLD) associated with accumulation of TAR-DNA binding protein of 43 kDA (TDP-43) inclusions (FTLD-TDP). Homozygous GRN mutations are linked to neuronal ceroid lipofuscinosis (NCL) (Townley, et al., Neurology, 2018 Jun. 12; 90 (24): 1127).

Mutations in the progranulin gene have recently been identified as a cause of about 5% of all FTD, including some sporadic cases. Recent studies using mouse models have defined the expression of progranulin in the brain (Petkau et al., 2010). Progranulin is expressed late in neurodevelopment, localizing with markers of mature neurons. Progranulin is expressed in neurons in most brain regions, with highest expression in the thalamus, hippocampus, and cortex. Microglia cells also express progranulin, and the level of expression is upregulated by microglial activation. Around 70 different progranulin gene mutations have been identified in FTD and all reduce progranulin levels or result in loss of progranulin function.

There is therefore an urgent need for therapeutic agents which can increase the expression and/or activity of progranulin.

For gapmer antisense oligonucleotides, mesylphosphoramidate modifications have been shown to improve the therapeutic index and duration of effect (Anderson et al., Nucleic acids research, 2021), while gapmer antisense oligonucleotide mesylphosphoramidate modifications have also been shown to be able to greatly reduce both immune stimulation and cytotoxicity (Anderson et al., Nucleic acids research, 2021). Mesylphosphoramidate linkage modifications can include methanesulfonyl phosphoramidate internucleotide linkages where, unlike other phosphoramidate and alkylphosphonate linkages, a negative charge is retained on the phosphate backbone.

Mesylphosphoramidate oligonucleotides can also act as splice-switching agents. However previous studies have not shown improved splice switching, as evaluation of mesylphosphoramidate oligonucleotide splice-switching activity in spinal muscular atrophy patient-derived fibroblasts revealed no significant difference in splice-switching efficacy between 2′-MOE mesyl oligonucleotides and the corresponding phosphorothioate (nusinersen) oligonucleotides (Hammond et al., Nucleic Acid Therapeutics, 2021).

### SUMMARY OF INVENTION

A splice variant of progranulin which retains the 5′ part of Intron 1 is expressed in the brain such as in neurons or microglia cells (Capell et al. The Journal of Biological Chemistry, 2014, 289 (37), 25879-25889). This splice variant include the 5′ most 271 nucleotides of intron 1, which totals 3823 nucleotides. The 271 nucleotide fragment of intron 1 includes two AUG sites upstream of the canonical downstream AUG (open reading frame) in exon 2. Translation from these two upstream AUG sites will not encode the progranulin protein, and due to premature termination codons the transcript may undergo non-sense mediated mRNA decay (NMD).

WO2020/191212 describes specific oligonucleotides which can target the progranulin mRNA.

Previously, the inventors have determined that reducing the splice variant which retains the 5′ part of intron 1 increases the Exon1 and Exon2 splice variant and further increases progranulin protein expression. This led to the invention of antisense oligonucleotides of progranulin. These antisense oligonucleotides are capable of altering the splicing pattern of progranulin, In particular the antisense oligonucleotides may up-regulate expression of the Exon1-Exon2 progranulin splice variant, reducing production of the progranulin Intron1-Exon2 splice variant which retains the 5′ part of intron 1, increasing the expression of the progranulin protein. These antisense oligonucleotides could be described as modulators of progranulin splicing, or as agonists of progranulin Exon1-Exon 2 and may be used to restore or enhance expression of the progranulin Exon1-Exon2 splice variant in cells.

The inventors have now surprisingly determined that this effect can be increased by including one or more methanesulfonyl phosphoramidate internucleotide linkages within the antisense oligonucleotide or contiguous nucleotide sequence thereof.

The present invention provides an antisense oligonucleotide, wherein the antisense oligonucleotide is 8-40 nucleotides in length and comprises a contiguous nucleotide sequence of 8-40 nucleotides in length which is complementary, such as fully complementary, to a splice regulation site of the human progranulin pre-mRNA transcript, wherein the contiguous nucleotide sequence comprises one or more methanesulfonyl phosphoramidate internucleotide linkages.

The invention provides an antisense oligonucleotide, wherein the antisense oligonucleotide is 8-40 nucleotides in length and comprises a contiguous nucleotide sequence of 8-40 nucleotides in length which is complementary, such as fully complementary, to a splice regulation site of the exon 1, intron 1 and exon 2 sequence of the human progranulin pre-mRNA transcript, wherein the contiguous nucleotide sequence comprises one or more methanesulfonyl phosphoramidate internucleotide linkages.

The invention provides an antisense oligonucleotide, wherein the antisense oligonucleotide is 8-40 nucleotides in length and comprises a contiguous nucleotide sequence of 8-40 nucleotides in length which is complementary, such as fully complementary, to a human progranulin pre-mRNA transcript that comprises the exon1, intron 1 and exon 2 sequence of the human progranulin pre-mRNA transcript (SEQ ID NO: 1), wherein the contiguous nucleotide sequence comprises one or more methanesulfonyl phosphoramidate internucleotide linkages.

The progranulin exon 1, intron 1 and exon 2 sequence is shown below as SEQ ID NO: 1. The progranulin exon1 sequence (in capital letters) corresponds to genome Ensemble (www.ensemble.org) chromosome 17 position 44,345,123; to position 44,345,334. Intron 1 corresponds to genome Ensemble chromosome 17 position 44,345,335 to 44,349,157 and Exon 2 sequence (in capital letters) corresponds to genome Ensemble chromosome 17 position 44,349,158 to position 44,349,302.

Exon 1, intron 1 and exon 2 sequence of the human progranulin pre-mRNA (SEQ ID NO: 1):
GGCGAGAGGAAGCAGGGAGGAGAGTGATTTGAGTAGAAAAGAAACACAGCATTCCAG
GCTGGCCCCACCTCTATATTGATAAGTAGCCAATGGGAGCGGGTAGCCCTGATCCCTG
GCCAATGGAAACTGAGGTAGGCGGGTCATCGCGCTGGGGTCTGTAGTCTGAGCGCTA
CCCGGTTGCTGCTGCCCAAGGACCGCGGAGTCGGACGCAGgtaggagagcggccgcgcagacc
tctcgcctgctcctgcccaggggcccgccagggccatgtgagcttgaggttcccctggagtctcagccggagacaacagaaga
accgcttactgaaactccttgggggttctgatacactagggggagttttatgggaaagaggaagcagtaattgcagtgacgcccc
gttagaaggggctttctacctccccagcattcccccaaagcagggaccacaccattcttgacccagctccacccctgtcggtaggt
gctggcttcttcccctctcctggtggtggtgggtggttcccgcggcggcctggagccggaggggcgcgcgaccctgggctggga
gctccgagggcctgggaacgagacctgagaccttggcttctcgaaggtagtagggacttgggagtggtgactgaacctggtctg
gctcctccttacttcctcttgttgcgggtgggacgagctagcttccgcctctcccagccactttttcctgctcatttgcagctaggttggct
ccccttttgggaatttcctctccccttggcactcggagttggggggtgccacctagtggaagataacggagctagggtcttgaaga
ggctgctgtcccctctggctgttttggcggtgtagggtggcatgagagactgcgactcgcctcctcatccctgtttctgtatgcgagtg
cttgtattcagtagaagcatacactatactccctcaatttagggtaaacaggaggggccacatgcacaggtaattcaccagggag
ccgaacactcctgtgcagacagactccccttcccagcaagccatggcagcggacagcctgctgagaacacccaggaagcag
gcggtgccagctgcaggtgctttgcctgggagctgtggggctgaggagagggtccactgtccaggaccagtgaacttcatcctta
tctgtccaggaggtggcctcttggggatgctgagttaggggaggggcacttgaggaaagccaggtggagcagagaggatgtg
agtgactgggtgggtgagatttcctgcccctccccccgcagtggtatccacacctagactcgtggggtaactgaggcacagaca
gagagcaacttctcaggccctcacagttggcaattctaggattaggacccaagtgcgattttcaggcagtccctgtaccctgtttctg
ttgtacctgttgcaccattcccaggcactgcccatcgtgccactagtgatatgaacccaggtccaatacgctctggggccatcaaa
gcctgacgtcaccatgacctgatgtgtgacgtgttataggtgtcccttggtatcttcacggaactggttccaggaccccaaaatctgt
gggtgctcaagcccctgagataaaatggtgtaatatttgcatataacctatacatactttaaatcatttctagattacttatacctaata
caatggaaatgacatgtcggctgggcgtggtggctcatgcctgtaatcccaccactttgggaggccgtggcaggtggatcacctg
aggtctggagtttgagaccagcctgaccaacatggtgaaacccccatctctactaaaaatacaaaaattagccaggtgtggtag
cgcacacctataatcccacctacttgggaggctgaggcaggagaattgcttgaacctgggaggcggagttcgcagtaagctga
gatcgcgccactgtactacagcctgggtgacagagcaggactccatctcaaaaaaaaaagagaaaaagaaaaagaaatgc
catgtaaatagttgtgatcctgaattgtttagggaataataagaaagaactatctgtagatgttcagtatagatgcacccatcgtaag
cctaactacattgtataactcagcaacgatgtaacattttcaggggtttttttgttttgttttttgagacagaatctcagtctcactctgtc
acccaggctggagtatgttggcgtgatctctgctcactgcaacctccacctcctgggctcaagcgattctcctgcctcagcctcttgagt
agctgggattgcaggtgtgcgctaccacgcatggctaatttttgtatttttaatagagatggggttttaccacgttggtcaggctggtctt
gaactcctgaccttgggatccgcccacctgggcctcccaaagtgctgggattacaggcgttagccaccgcgcccaatatattttga
tccctggttggatatggagggctgactgtacttaacatctctaagcttcagtttcctcctttaaaataaaggtgtggctgggtgtggtgg
ttcaagcctgtaatcccagcacttagggaggctgaggtgggtggatcagctgaggtcaggagttcaagaccagcctgaccaata
tggtgaaaccccctctctgctaaaaatacaaaaattagccaggcgtggtggcgagcgcctgtagtcccagctacttgcttgaactt
gggaggcagaggttgcagtgagctgagatcgtgccactgaactcgagcatgggcaacagagcaagactgtctcaaaaaaaa
aaaaaaaaagggggtgagcagacgtggtggcacgctcccacagtcccagctacttagtaggaggccaaggttggaggattg
cttgatcccaggagtctgagtccagcctgggcaacatggcaatacctcatctctaaaaataaaataaaagtaaaggtattaattac
tactttggatggttgttgcaaagaaatatatataaaataatggagagtcttgtaactggctcccaagaggctcaacagacattactgt
ttttgcttcttcattatgagttacctctctggccaccccactgaactagctgggctagctgagcctgggagaagagttgtttaggaagt
gagaggctgctctccacagagactcaaggctcagttcctcctggtgactcagatgggcagcccagtgggcacacgtggtctctct
ccacatgtggctgagtttcacttccagaatagatggagaggcaagggcagggtttagcatgcttgaggaatctcagagggccct
ggtggtgtgggggaccctcagaacacaggtgtctcaagggctgacccagcttctgtgtccttttctctgggtgaggaggggacatt
catgggcagatggtgacctctggggaaggcagcccagactccactggccaccatatttcctttttcacaactttctcacccctgtggt
ttcccatgtcatcatgtggccgcttcccgcaaggccttagcggggtgcaggtatgaacatagtgtcaggcaaggaggcatctgga
ggggaaccctggcttttcctggggggactccctccctgcaccctagccctgtcctctcccatggctactgatgccttcccctcacccc
agaggtggcccacatctgcacagatcagacccacaaaaatcacgtcttcctgactctcataagcctgcccagtgaggcccagg
cattaggccatgtgctggggactcagacccacacatatacgcatgtcagcattcatgcttacaggtccgcacatgctggggcaag
tgtcacacacggggcgctgtaggaagctgactctcagcccctgcagatttctgcctgcctggacagggaggtgttgagaaggctc
aggcagtcctgggccaggaccttggcctggggctagggtactgagtgaccctagaatcaagggtggcgtgggcttaagcagttg
ccagacgttccttggtactttgcagGCAGACCATGTGGACCCTGGTGAGCTGGGTGGCCTTAACAG
CAGGGCTGGTGGCTGGAACGCGGTGCCCAGATGGTCAGTTCTGCCCTGTGGCCTGCT
GCCTGGACCCCGGAGGAGCCAGCTACAGCTGCTGCCGTCCCCTTCTG

The invention provides an antisense oligonucleotide, wherein the antisense oligonucleotide is 8-40 nucleotides in length and comprises a contiguous nucleotide sequence of at least 12 nucleotides in length which is complementary, such as fully complementary, to a splice regulation site of the human progranulin pre-mRNA, wherein the contiguous nucleotide sequence comprises one or more methanesulfonyl phosphoramidate internucleotide linkages.

The invention provides an antisense oligonucleotide, wherein the antisense oligonucleotide is 8-40 nucleotides in length and comprises a contiguous nucleotide sequence of 12-16 nucleotides in length which is complementary, such as fully complementary, to a splice regulation site of the human progranulin pre-mRNA, wherein the contiguous nucleotide sequence comprises one or more methanesulfonyl phosphoramidate internucleotide linkages.

The invention provides an antisense oligonucleotide, wherein the antisense oligonucleotide is 12-16 nucleotides in length and comprises a contiguous nucleotide sequence of 12-16 nucleotides in length which is complementary, such as fully complementary, to a splice regulation site of the human progranulin pre-mRNA, wherein the contiguous nucleotide sequence comprises one or more methanesulfonyl phosphoramidate internucleotide linkages.

The invention provides an antisense oligonucleotide, wherein the antisense oligonucleotide is 8-40 nucleotides in length and comprises a contiguous nucleotide sequence of 12-18 nucleotides in length which is complementary, such as fully complementary, to a splice regulation site of the human progranulin pre-mRNA, wherein the contiguous nucleotide sequence comprises one or more methanesulfonyl phosphoramidate internucleotide linkages.

The invention provides an antisense oligonucleotide, wherein the antisense oligonucleotide is 12-18 nucleotides in length and comprises a contiguous nucleotide sequence of 12-18 nucleotides in length which is complementary, such as fully complementary, to a splice regulation site of the human progranulin pre-mRNA, wherein the contiguous nucleotide sequence comprises one or more methanesulfonyl phosphoramidate internucleotide linkages.

The invention provides an antisense oligonucleotide, wherein the antisense oligonucleotide is 8-40 nucleotides in length and comprises a contiguous nucleotide sequence of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length which is complementary, such as fully complementary, to a splice regulation site of the human progranulin pre-mRNA, wherein the contiguous nucleotide sequence comprises one or more methanesulfonyl phosphoramidate internucleotide linkages.

The invention provides an antisense oligonucleotide, wherein the antisense oligonucleotide is 8-40 nucleotides in length and comprises a contiguous nucleotide sequence of 8-40 nucleotides in length which is complementary, such as fully complementary, to a nucleotide sequence comprised within SEQ ID NO: 1, wherein the contiguous nucleotide sequence comprises one or more methanesulfonyl phosphoramidate internucleotide linkages.

The invention provides an antisense oligonucleotide, wherein the antisense oligonucleotide is 8-40 nucleotides in length and comprises a contiguous nucleotide sequence of 8-40 nucleotides in length which is complementary, such as fully complementary, to a nucleotide sequence comprised within nucleotides 449-466 of SEQ ID NO: 1, wherein the contiguous nucleotide sequence comprises one or more methanesulfonyl phosphoramidate internucleotide linkages.

The invention provides an antisense oligonucleotide, wherein the antisense oligonucleotide is 8-40 nucleotides in length and comprises a contiguous nucleotide sequence of 8-40 nucleotides in length which is complementary, such as fully complementary, to SEQ ID NO: 39, wherein the contiguous nucleotide sequence comprises one or more methanesulfonyl phosphoramidate internucleotide linkages.

SEQ ID NO: 39 is a target site having the sequence: ACCACACCATTCTTGACC

The antisense oligonucleotide may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the antisense oligonucleotide is 8-40, 12-40, 12-20, 10-20, 14-18, 12-18 or 16-18 nucleotides in length.

The contiguous nucleotide sequence may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments, the contiguous nucleotide sequence is of a length of at least 12 nucleotides in length, such as 12-16 or 12-18 nucleotides in length.

In some embodiments, the contiguous nucleotide sequence is the same length as the antisense oligonucleotide.

In some embodiments the antisense oligonucleotide consists of the contiguous nucleotide sequence.

In some embodiments the antisense oligonucleotide is the contiguous nucleotide sequence.

In some embodiments, the contiguous nucleotide sequence is fully complementary to a nucleotide sequence comprised within SEQ ID NO: 1.

In some embodiments, the contiguous nucleotide sequence is fully complementary to a nucleotide sequence comprised within nucleotides 449-466 of SEQ ID NO: 1.

In some embodiments, the contiguous nucleotide sequence is fully complementary to SEQ ID NO: 39.

In some embodiments, the contiguous nucleotide sequence is a sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38, or at least 8 contiguous nucleotides thereof.

In some embodiments, the contiguous nucleotide sequence is a sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38, or at least 9 contiguous nucleotides thereof.

In some embodiments, the contiguous nucleotide sequence is a sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38, or at least 10 contiguous nucleotides thereof.

In some embodiments, the contiguous nucleotide sequence is a sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38, or at least 11 contiguous nucleotides thereof.

In some embodiments, the contiguous nucleotide sequence is a sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38, or at least 12 contiguous nucleotides thereof.

In some embodiments, the contiguous nucleotide sequence is a sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38, or at least 13 contiguous nucleotides thereof.

In some embodiments, the contiguous nucleotide sequence is a sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38, or at least 14 contiguous nucleotides thereof.

In some embodiments, the contiguous nucleotide sequence is a sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38, or at least 15 contiguous nucleotides thereof.

In some embodiments the contiguous nucleotide sequence is selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38.

In some embodiments the contiguous nucleotide sequence is selected from the group consisting of SEQ ID NO: 5, SEQ ID NO: 6, SEQ ID NO: 7, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 30, SEQ ID NO: 31, SEQ ID NO: 34, SEQ ID NO: 35, SEQ ID NO: 36 and SEQ ID NO: 37.

In some embodiments the contiguous nucleotide sequence is selected from the group consisting of SEQ ID NO: 11, SEQ ID NO: 15, SEQ ID NO: 16, SEQ ID NO: 28, SEQ ID NO: 31 and SEQ ID NO: 35.

In some embodiments the contiguous nucleotide sequence is SEQ ID NO: 11.

In some embodiments the contiguous nucleotide sequence is SEQ ID NO: 15.

In some embodiments the contiguous nucleotide sequence is SEQ ID NO: 16.

In some embodiments the contiguous nucleotide sequence is SEQ ID NO: 28.

In some embodiments the contiguous nucleotide sequence is SEQ ID NO: 31.

In some embodiments the contiguous nucleotide sequence is SEQ ID NO: 35.

In some embodiments, the contiguous nucleotide sequence comprises 1, 2, 3, 4, 5, 6, 7, 8, 9, 10, 11, 12, 13, 14, 15, 16, 17 or more methanesulfonyl phosphoramidate internucleotide linkages.

In some embodiments, the contiguous nucleotide sequence comprises 1 or more methanesulfonyl phosphoramidate internucleotide linkages.

In some embodiments, the contiguous nucleotide sequence comprises 2 or more methanesulfonyl phosphoramidate internucleotide linkages.

In some embodiments, the contiguous nucleotide sequence comprises 3 or more methanesulfonyl phosphoramidate internucleotide linkages.

In some embodiments, the contiguous nucleotide sequence comprises 4 or more methanesulfonyl phosphoramidate internucleotide linkages.

In some embodiments, the contiguous nucleotide sequence comprises 5 or more methanesulfonyl phosphoramidate internucleotide linkages.

In some embodiments, the contiguous nucleotide sequence comprises 6 or more methanesulfonyl phosphoramidate internucleotide linkages.

In some embodiments, the contiguous nucleotide sequence comprises 7 or more methanesulfonyl phosphoramidate internucleotide linkages.

In some embodiments, the contiguous nucleotide sequence comprises 8 or more methanesulfonyl phosphoramidate internucleotide linkages.

In some embodiments, the contiguous nucleotide sequence comprises 9 or more methanesulfonyl phosphoramidate internucleotide linkages.

In some embodiments, the contiguous nucleotide sequence comprises 10 or more methanesulfonyl phosphoramidate internucleotide linkages.

In some embodiments, the contiguous nucleotide sequence comprises 11 or more methanesulfonyl phosphoramidate internucleotide linkages.

In some embodiments, the contiguous nucleotide sequence comprises 12 or more methanesulfonyl phosphoramidate internucleotide linkages.

In some embodiments, the contiguous nucleotide sequence comprises 13 or more methanesulfonyl phosphoramidate internucleotide linkages.

In some embodiments, the contiguous nucleotide sequence comprises 14 or more methanesulfonyl phosphoramidate internucleotide linkages.

In some embodiments, the contiguous nucleotide sequence comprises 15 or more methanesulfonyl phosphoramidate internucleotide linkages.

In some embodiments, the contiguous nucleotide sequence comprises 16 or more methanesulfonyl phosphoramidate internucleotide linkages.

In some embodiments, the contiguous nucleotide sequence comprises 17 or more methanesulfonyl phosphoramidate internucleotide linkages.

In some embodiments, the contiguous nucleotide sequence comprises one or more phosphorothioate internucleotide linkages.

In some embodiments, the contiguous nucleotide sequence comprises multiple phosphorothioate internucleotide linkages, such as 1, 2, 3, 4, 5, 6, 7, 8, 9, 10 or more phosphorothioate internucleotide linkages.

In some embodiments, at least about 75%, at least about 80%, at least about 85%, at least about 90%, at least about 95%, or about 100% of the internucleotide linkages of the contiguous nucleotide sequence are modified.

In some embodiments, all of the internucleotide linkages positioned between the nucleotides of the contiguous nucleotide sequence are modified.

In some embodiments, all the internucleotide linkages present in the antisense oligonucleotide are selected from phosphorothioate internucleotide linkages and methanesulfonyl phosphoramidate internucleotide linkages.

In some embodiments, the antisense oligonucleotide, or contiguous nucleotide sequence thereof, comprises one or more modified nucleosides.

In some embodiments, the contiguous nucleotide sequence comprises one or more 2′-O-methoxyethyl-RNA (2′-MOE) nucleosides.

In some embodiments, the contiguous nucleotide sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32, 33, 34, 35, 36, 37, 38, 39 or 40 2′-O-methoxyethyl-RNA (2′-MOE) nucleosides.

In some embodiments, the contiguous nucleotide sequence comprises at least 10%, at least 20%, at least 30%, at least 40%, at least 50%, at least 60%, at least 70%, at least 80%, at least 90% or 100% 2′-O-methoxyethyl-RNA (2′-MOE) nucleosides.

In some embodiments, the nucleosides of the contiguous nucleotide sequence are 2′-O-methoxyethyl-RNA (2′-MOE) nucleosides.

The invention provides for an antisense oligonucleotide which is isolated, purified or manufactured.

In some embodiments, the antisense oligonucleotide is or comprises an antisense oligonucleotide mixmer or totalmer. In some embodiments, the contiguous nucleotide sequence is a mixmer or a totalmer.

The invention provides for a conjugate comprising the antisense oligonucleotide according to the invention, and at least one conjugate moiety covalently attached to said antisense oligonucleotide.

The invention provides an antisense oligonucleotide covalently attached to at least one conjugate moiety.

The invention provides for a pharmaceutically acceptable salt of the antisense oligonucleotide according to the invention, or the conjugate according to the invention.

The invention provides for an antisense oligonucleotide according to the invention wherein the antisense oligonucleotide is in the form of a pharmaceutically acceptable salt.

In some embodiments the pharmaceutically acceptable salt may be a sodium salt, a potassium salt or an ammonium salt.

The invention provides for a pharmaceutically acceptable sodium salt of the antisense oligonucleotide according to the invention, or the conjugate according to the invention.

The invention provides for a pharmaceutically acceptable potassium salt of the antisense oligonucleotide according to the invention, or the conjugate according to the invention.

The invention provides for a pharmaceutically acceptable ammonium salt of the antisense oligonucleotide according to the invention, or the conjugate according to the invention.

The invention provides for a pharmaceutical composition comprising the antisense oligonucleotide of the invention, or the conjugate of the invention, and a pharmaceutically acceptable diluent, solvent, carrier, salt and/or adjuvant.

The invention provides for a pharmaceutical composition comprising the antisense oligonucleotide of the invention, or the conjugate of the invention, and a pharmaceutically acceptable salt. For example, the salt may comprise a metal cation, such as a sodium salt, a potassium salt or an ammonium salt.

The invention provides for a pharmaceutical composition according to the invention, wherein the pharmaceutical composition comprises the antisense oligonucleotide of the invention or the conjugate of the invention, or the pharmaceutically acceptable salt of the invention; and an aqueous diluent or solvent.

The invention provides for a solution, such as a phosphate buffered saline solution of the antisense oligonucleotide of the invention, or the conjugate of the invention, or the pharmaceutically acceptable salt of the invention. Suitably the solution, such as phosphate buffered saline solution, of the invention, is a sterile solution.

The invention provides for a method for enhancing the expression of the Exon1-Exon2 progranulin splice variant in a cell which is expressing progranulin, said method comprising administering an antisense oligonucleotide of the invention, or a conjugate of the invention, or a salt of the invention, or a pharmaceutical composition of the invention in an effective amount to said cell. In some embodiments the method is an in vitro method. In some embodiments the method is an in vivo method.

In some embodiments, the cell is either a human cell or a mammalian cell.

The invention provides for a method for treating or preventing progranulin haploinsufficiency or a related disorder, comprising administering a therapeutically or prophylactically effective amount of an antisense oligonucleotide of the invention, or a conjugate of the invention, or a salt of the invention, or a pharmaceutical composition of the invention to a subject suffering from or susceptible to progranulin haploinsufficiency or a related disorder.

The invention provides for a method for treating or preventing neurological disease, comprising administering a therapeutically or prophylactically effective amount of an antisense oligonucleotide of the invention, or a conjugate of the invention, or a salt of the invention, or a pharmaceutical composition of the invention to a subject suffering from or susceptible to neurological disease. In one embodiment the neurological disease may be a TDP-43 pathology.

The invention provides for an antisense oligonucleotide of the invention, for use as a medicament.

The invention provides for an antisense oligonucleotide of the invention, for use in therapy.

The invention provides for the antisense oligonucleotide of the invention or the conjugate of the invention, or the salt of the invention, or the pharmaceutical composition of the invention, for use as a medicament.

The invention provides the antisense oligonucleotide of the invention or the conjugate of the invention, or the salt of the invention, or the pharmaceutical composition of the invention for use in therapy.

The invention provides for the antisense oligonucleotide of the invention or the conjugate of the invention, or the salt of the invention, or the pharmaceutical composition of the invention for use in the treatment of a neurological disease. In one embodiment the neurological disease may be a TDP-43 pathology.

The invention provides for the antisense oligonucleotide of the invention or the conjugate of the invention, or the salt of the invention, or the pharmaceutical composition of the invention for use in the treatment or prevention of progranulin haploinsufficiency or a related disorder.

The invention provides for the use of the antisense oligonucleotide of the invention or the conjugate of the invention, or the salt of the invention, or the pharmaceutical composition of the invention, for the preparation of a medicament for treatment or prevention of a neurological disease. In one embodiment the neurological disease may be a TDP-43 pathology.

The invention provides for the use of the antisense oligonucleotide of the invention or the conjugate of the invention, or the salt of the invention, or the pharmaceutical composition of the invention, for the preparation of a medicament for treatment or prevention of progranulin haploinsufficiency or a related disorder.

In some embodiments the method, use, or antisense oligonucleotide for use, of the invention is for the treatment of frontotemporal dementia (FTD), neuropathologic frontotemporal lobar degeneration or neuroinflammation. In other embodiments the method, use, or antisense oligonucleotide for use, of the invention is for the treatment of amyotrophic lateral sclerosis (ALS), Alzheimer's disease, Parkinson's disease, Autism, Hippocampal sclerosis dementia, Down syndrome, Huntington's disease, polyglutamine diseases, spinocerebellar ataxia 3, myopathies or Chronic Traumatic Encephalopathy.

In one aspect the invention includes an oligonucleotide progranulin agonist having the structure:

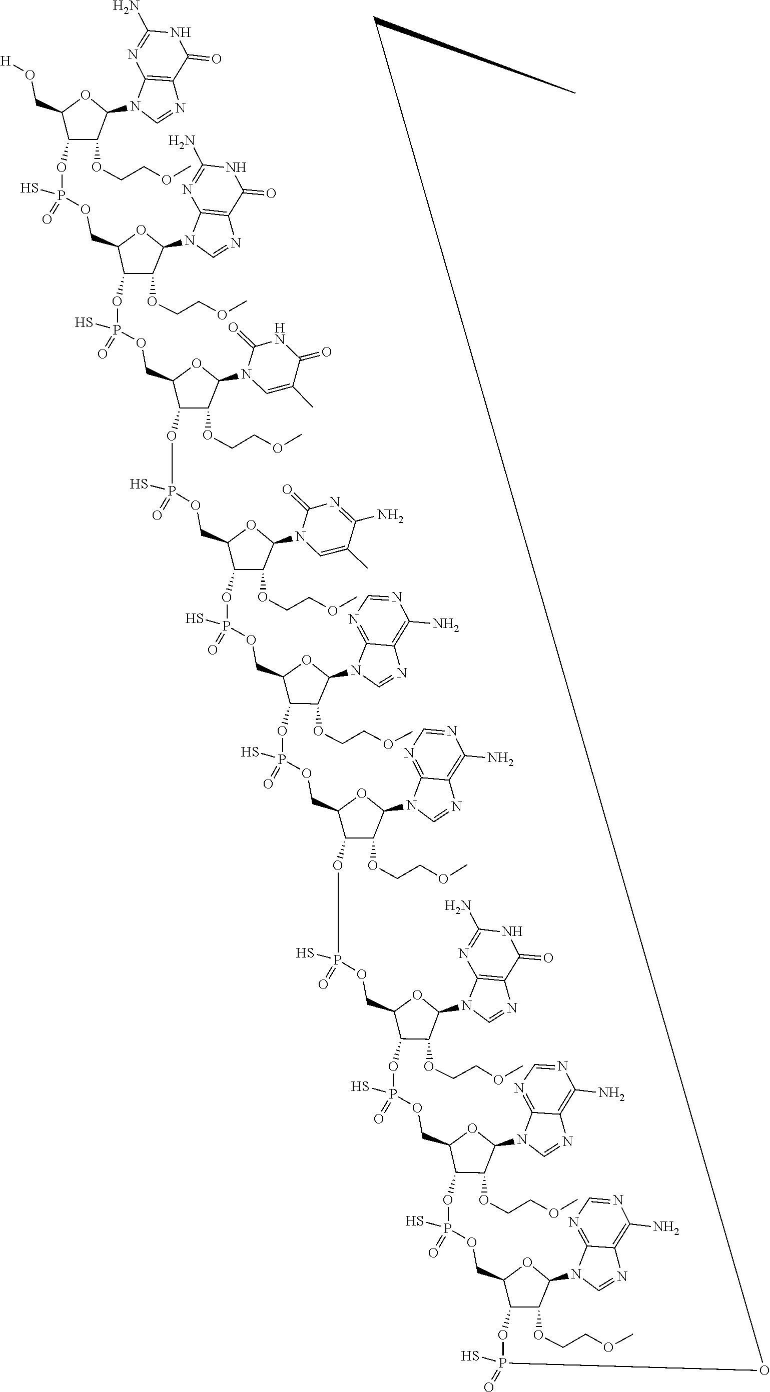

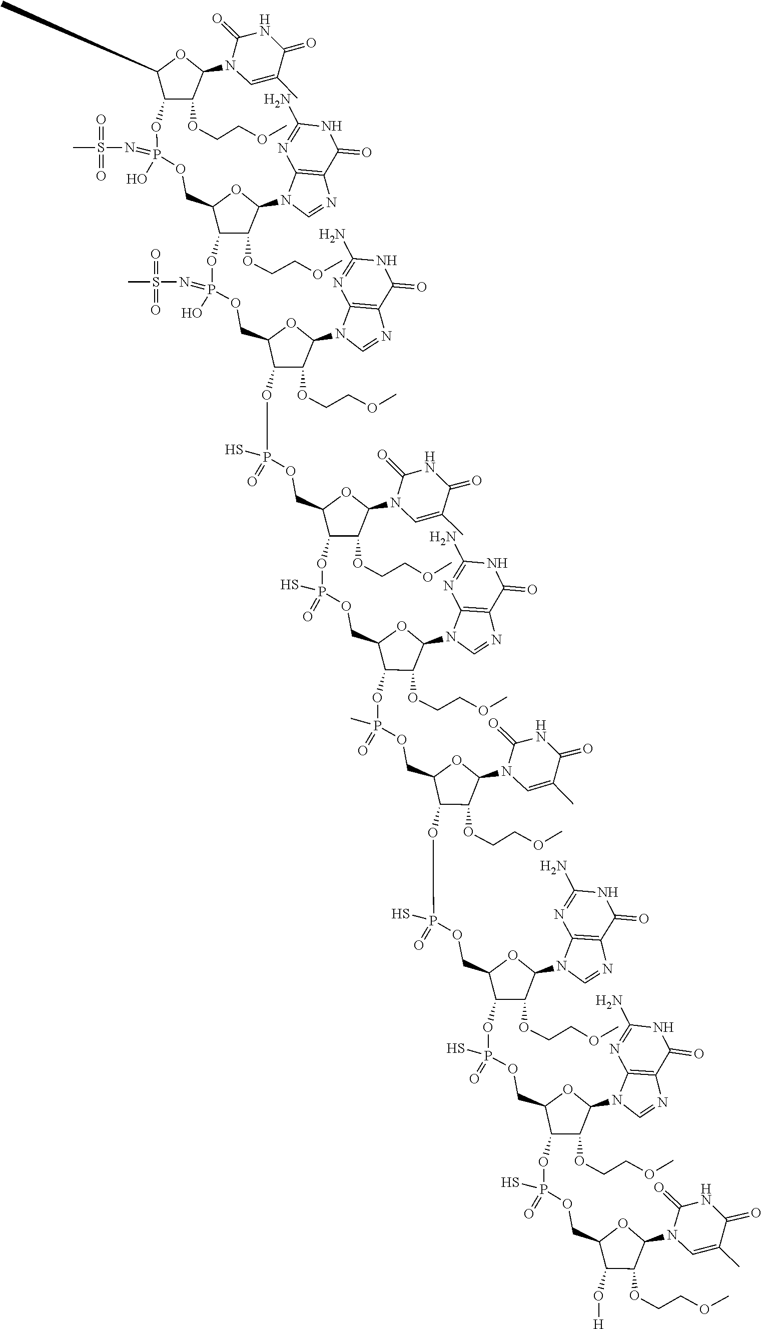

In one aspect the invention includes an oligonucleotide progranulin agonist having the structure:

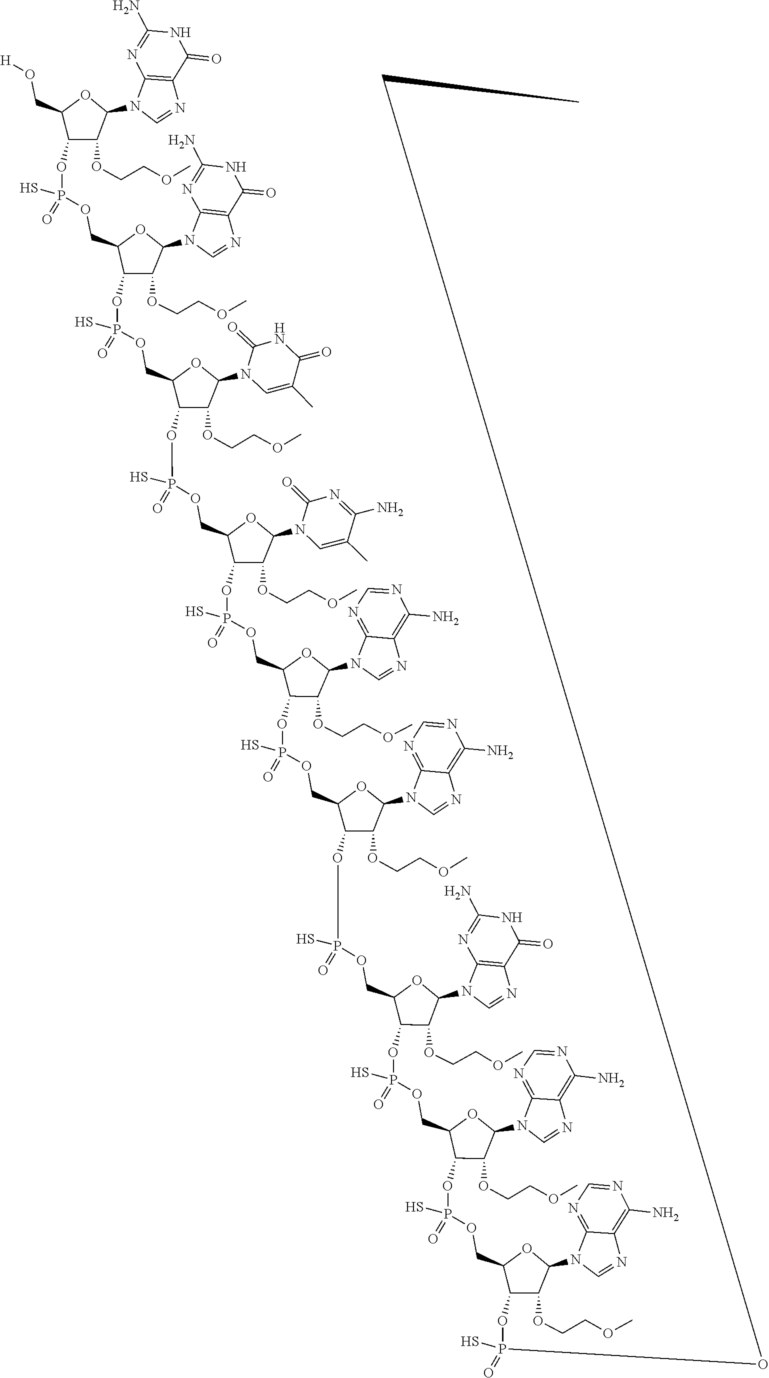

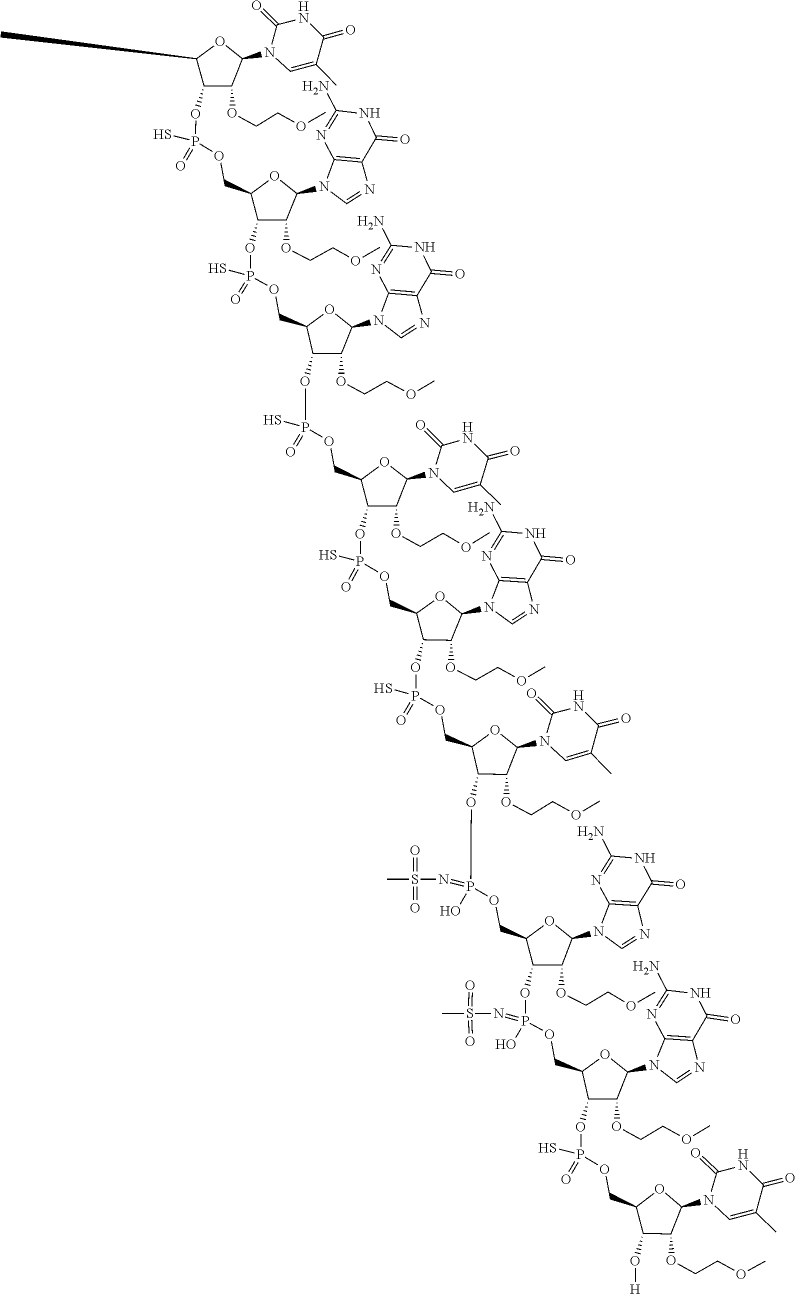

In one aspect the invention includes an oligonucleotide progranulin agonist having the structure:

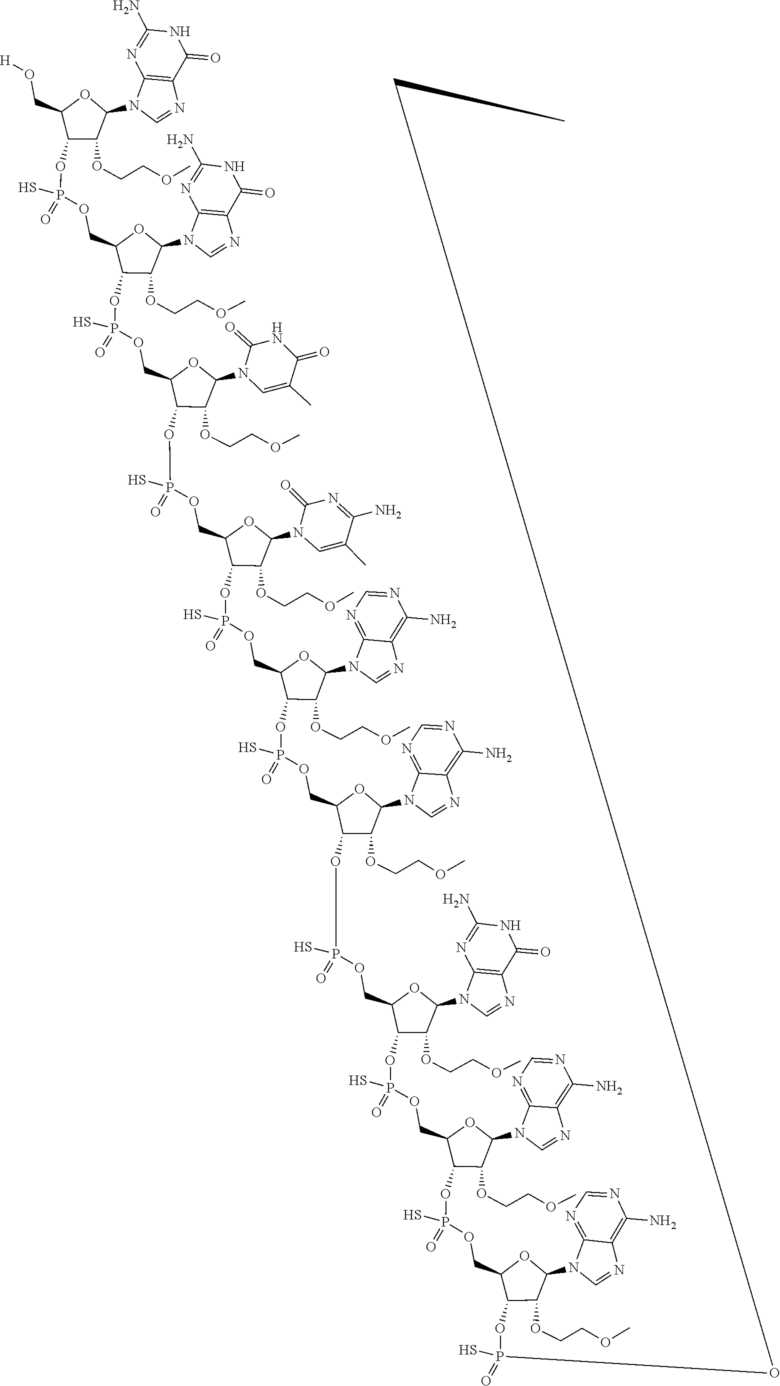

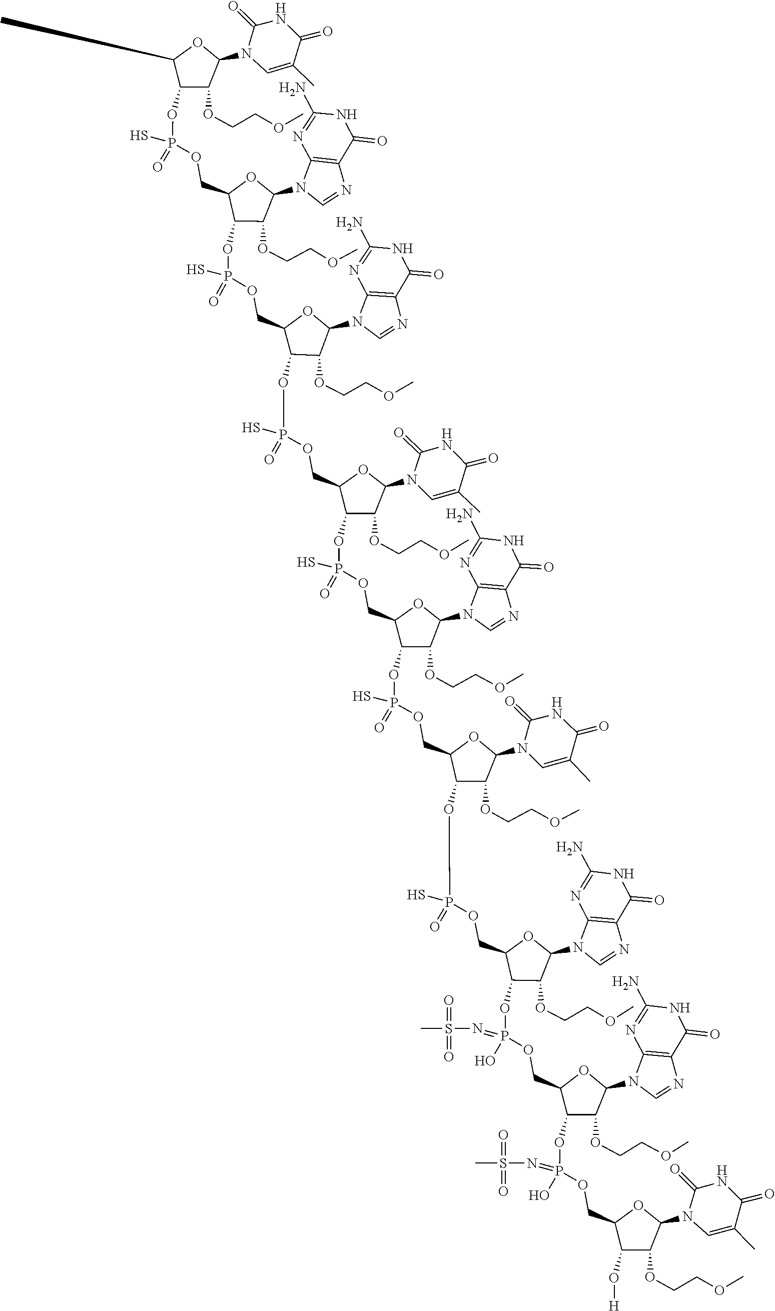

In one aspect the invention includes an oligonucleotide progranulin agonist having the structure:

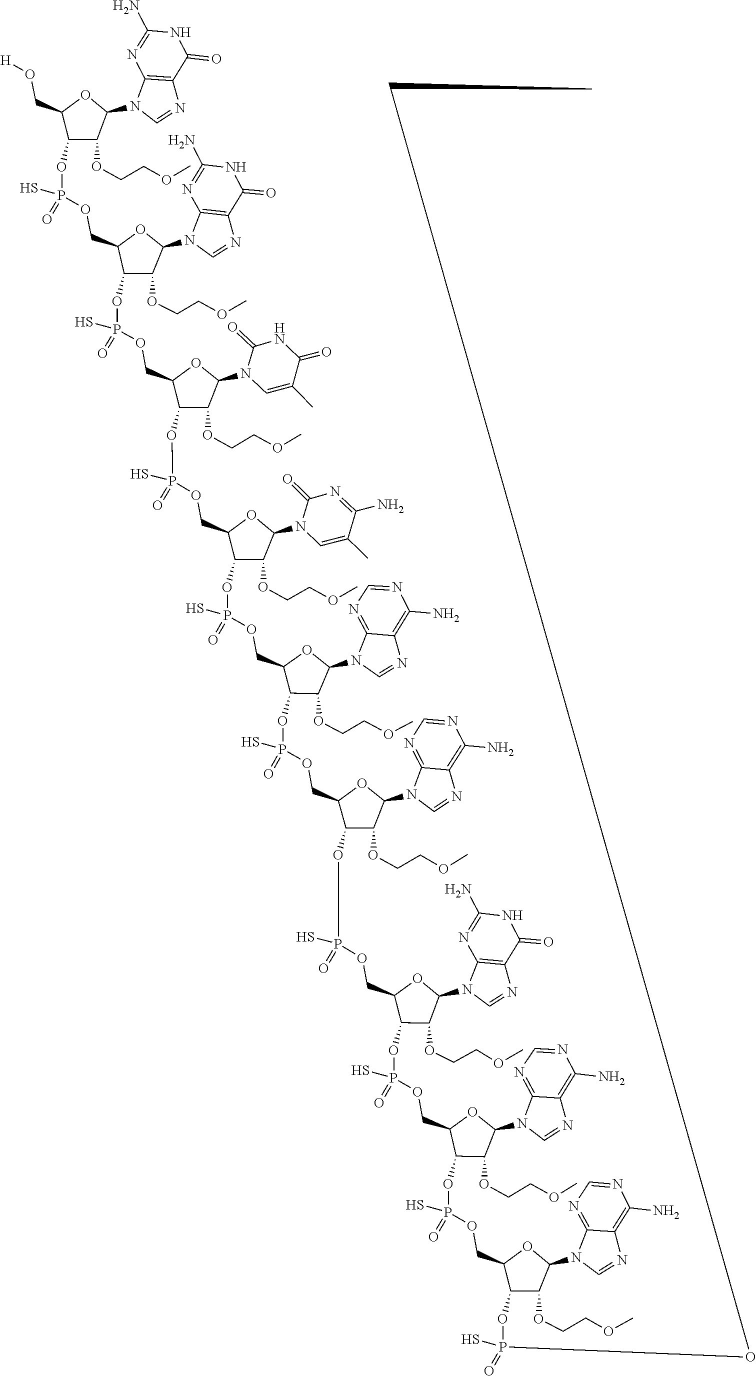

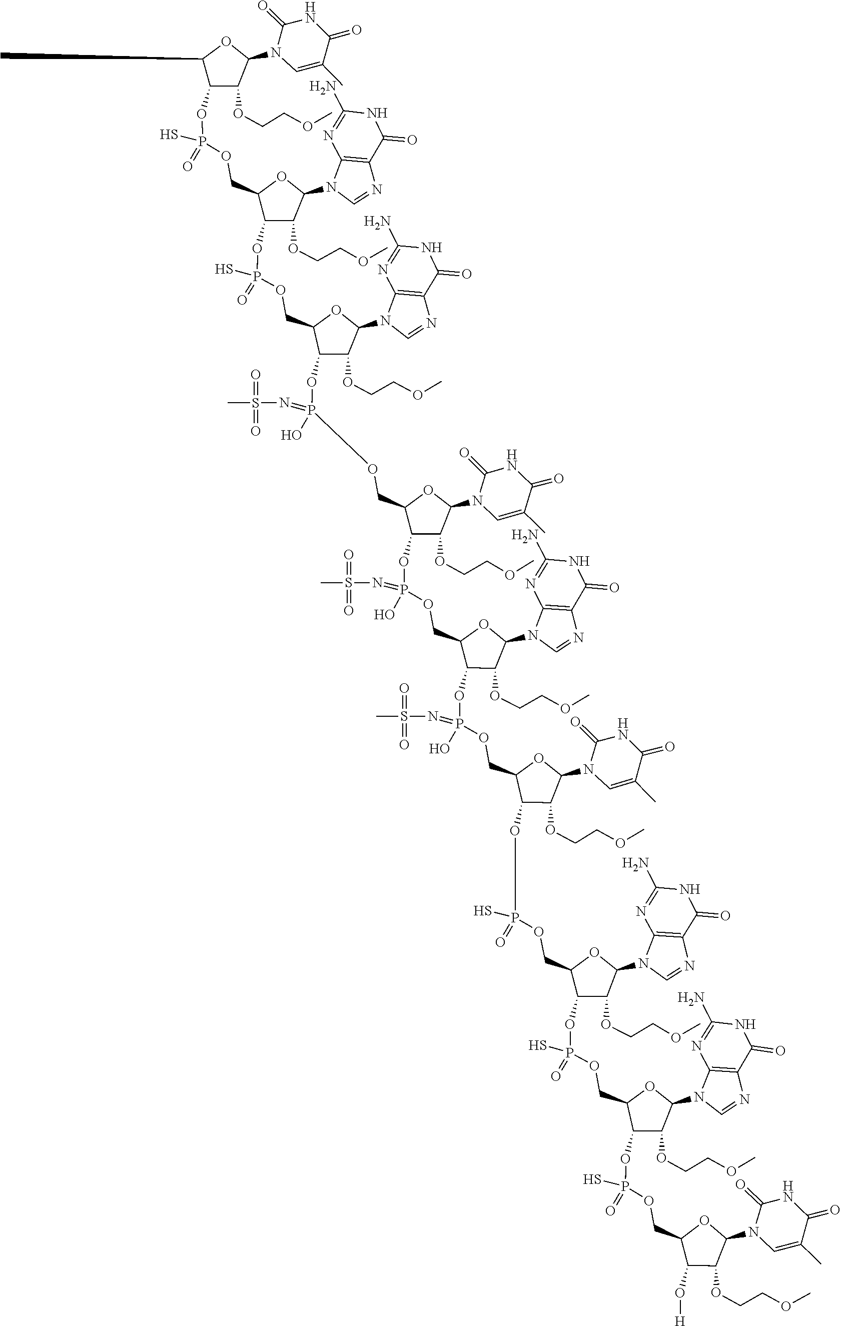

In one aspect the invention includes an oligonucleotide progranulin agonist having the structure:

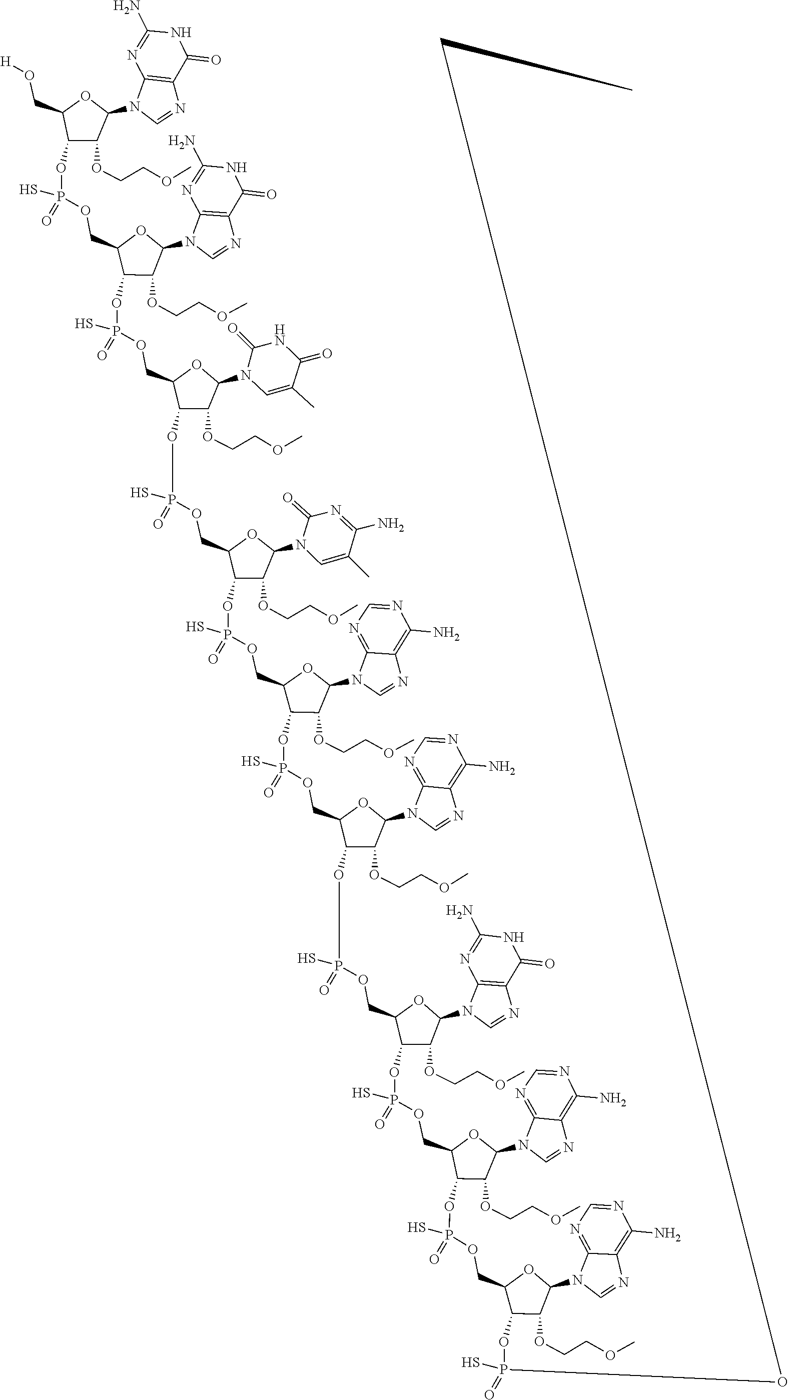

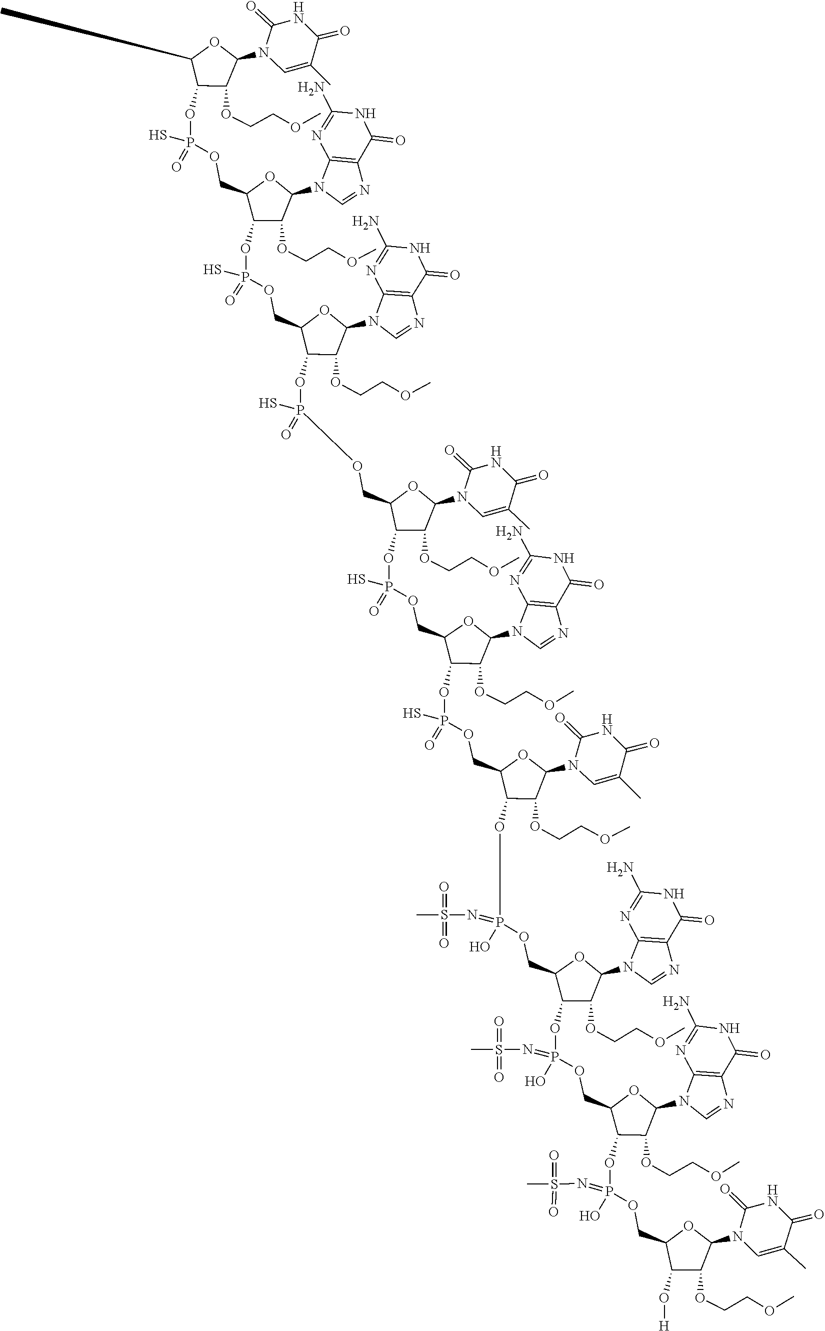

In one aspect the invention includes an oligonucleotide progranulin agonist having the structure:

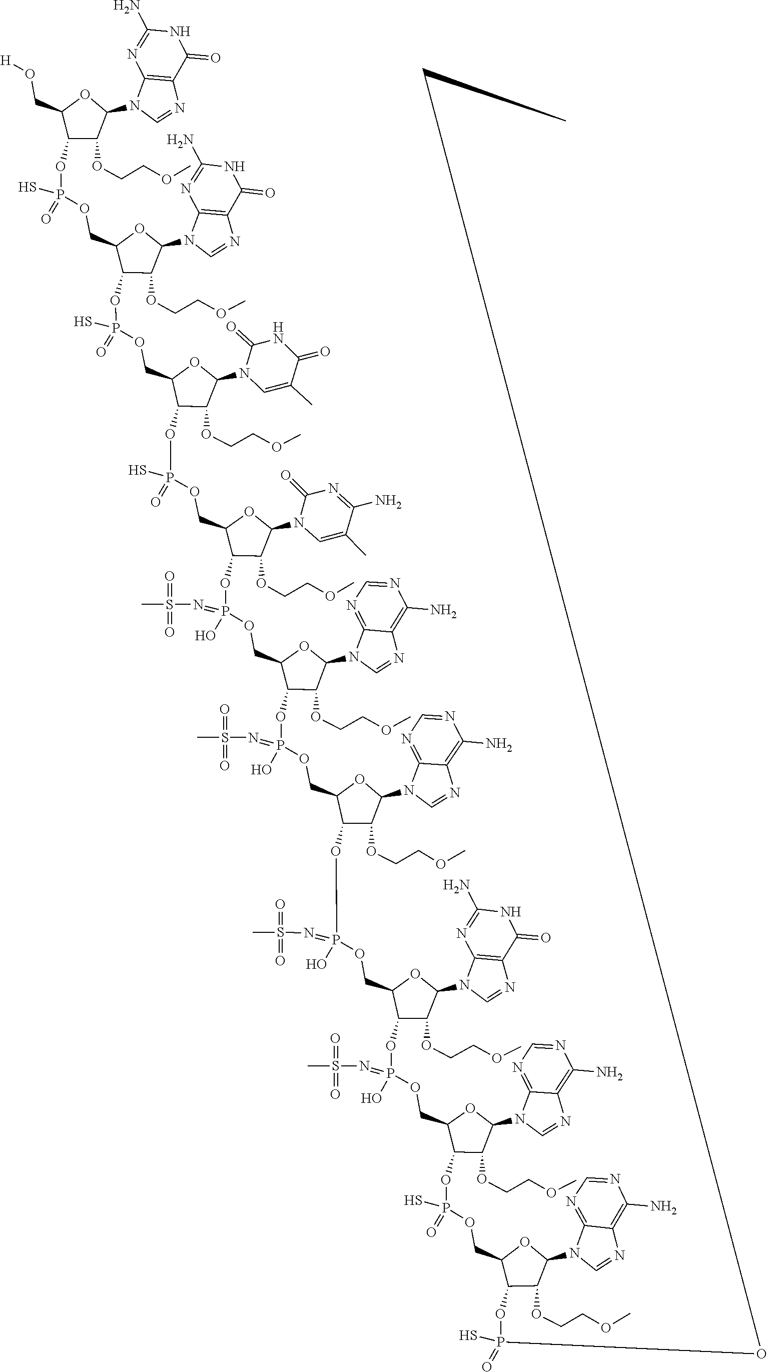

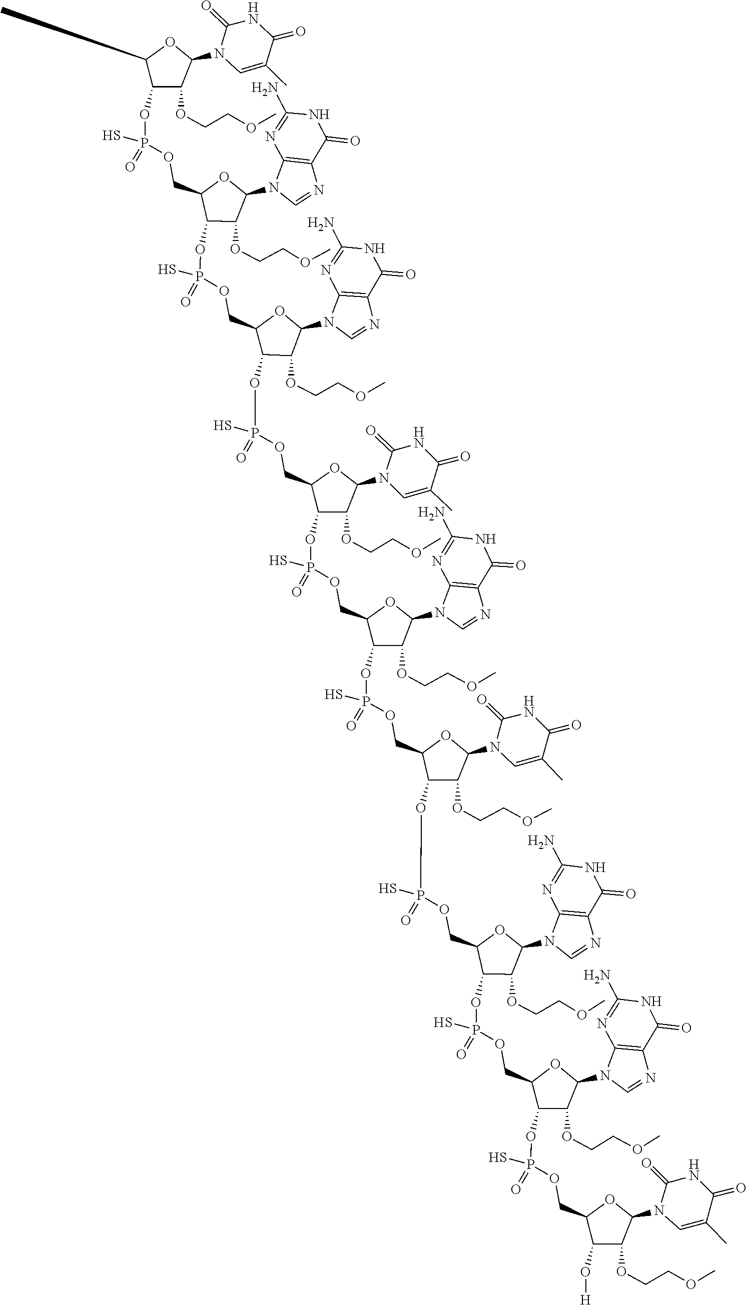

In another aspect the invention includes an antisense oligonucleotide wherein the oligonucleotide is the oligonucleotide compound GGTCAAGAATGGTGTGGT (SEQ ID NO: 11, SEQ ID NO: 15, SEQ ID NO: 16, SEQ ID NO: 28, SEQ ID NO: 31 or SEQ ID NO: 35), wherein all the nucleosides are 2′-O-methoxyethyl-RNA (2′-MOE) nucleosides, the C is 5-methyl cytosine and all internucleoside linkages are selected from phosphorothioate internucleoside linkages and methanesulfonyl phosphoramidate internucleoside linkages.

In another aspect the invention includes an antisense oligonucleotide wherein the oligonucleotide is the oligonucleotide compound GGTCAAGAATGGTGTGGT (SEQ ID NO: 2), wherein all the nucleosides are 2′-O-methoxyethyl-RNA (2′-MOE) nucleosides, the C is 5-methyl cytosine and all internucleoside linkages are methanesulfonyl phosphoramidate internucleoside linkages.

### BRIEF DESCRIPTION OF FIGURES

FIG. 1a: ddPCR data quantifying the abundance of the 5′ UTR splice variant with retention of intron1 in GRN mRNA after 5 days gymnosis in Microglia cells, relative to PBS treated cells. Grey bars quantify the abundance of the splice variant with retention of intron1 (Int1-Ex2) after treatment with 3 μM and black bars quantify the abundance of the splice variant with retention of intron1 (Int1-Ex2) after treatment with 10 μM.

FIG. 1b: ddPCR data quantifying the abundance of the 5′ UTR Exon1-Exon2 splice variants in GRN mRNA after 5 days gymnosis in Microglia cells, relative to PBS treated cells. Grey bars quantify the abundance of the splice variant Exon1-Exon2 (Ex1-Ex2) after treatment with 3 UM and black bars quantify the abundance of the splice variant Exon1-Exon2 (Ex1-Ex2) after treatment with 10 μM.

### DEFINITIONS

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers.

Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides.

The oligonucleotides of the invention are man-made, and are chemically synthesized, and are typically purified or isolated.

The oligonucleotides of the invention may comprise one or more modified nucleosides such as 2′-MOE nucleosides and may further comprise one or more further or additionally modified nucleosides such as 2′ sugar modified nucleosides.

The oligonucleotides of the invention may comprise one or more modified internucleoside linkages, such as one or more methanesulfonyl phosphoramidate internucleoside linkages and one or more phosphorothioate internucleoside linkages.

### Antisense Oligonucleotide

The term “antisense oligonucleotide” as used herein is defined as an oligonucleotide capable of modulating expression of a target gene by hybridizing to a target nucleic acid, in particular to a contiguous sequence on a target nucleic acid.

Antisense oligonucleotides are not essentially double stranded and are therefore not siRNAs or shRNAs.

The antisense oligonucleotides of the present invention may be single stranded. It is understood that single stranded oligonucleotides of the present invention can form hairpins or intermolecular duplex structures (duplex between two molecules of the same oligonucleotide), as long as the degree of intra or inter self-complementarity is less than approximately 50% across of the full length of the oligonucleotide.

In certain contexts the antisense oligonucleotides of the invention may be referred to as oligonucleotides.

In some embodiments, the single stranded antisense oligonucleotides of the invention may not contain RNA nucleosides.

Advantageously, the antisense oligonucleotides of the invention comprise one or more modified nucleosides or nucleotides, such as 2′ sugar modified nucleosides. Furthermore, in some antisense oligonucleotides of the invention, it may be advantageous that the nucleosides which are not modified are DNA nucleosides.

### Contiguous Nucleotide Sequence

The term “contiguous nucleotide sequence” refers to the region of the oligonucleotide which is complementary to a target nucleic acid, which may be or may comprise an oligonucleotide motif sequence. The term is used interchangeably herein with the term “contiguous nucleobase sequence”.

In some embodiments all the nucleosides of the oligonucleotide constitute the contiguous nucleotide sequence. The contiguous nucleotide sequence is the sequence of nucleotides in the oligonucleotide of the invention which is complementary to, and in some instances fully complementary to, the target nucleic acid or target sequence, or target site sequence. The terms “target nucleic acid”, “target sequence” and “target site sequence” may be used interchangeably to refer to the sequence bound by the contiguous nucleotide sequence.

In some embodiments the target sequence is SEQ ID NO: 1.

SEQ ID NO: 1 is the sequence of exon 1, intron 1 and exon 2 of the human progranulin pre-mRNA transcript.

In some embodiments the target sequence is or comprises nucleotides 449-466 of SEQ ID NO: 1.

In some embodiments the target sequence is or comprises SEQ ID NO: 39.

In some embodiments the target sequence is SEQ ID NO: 39.

In some embodiments the target sequence comprises SEQ ID NO: 39.

In some embodiments the oligonucleotide comprises the contiguous nucleotide sequence, and may optionally comprise further nucleotide(s), for example a nucleotide linker region which may be used to attach a functional group (e.g. a conjugate group) to the contiguous nucleotide sequence.

The nucleotide linker region may or may not be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

### Splice Regulation Site

The term “splice regulation site” as used herein is defined as a site within a pre-mRNA transcript which affects splicing of that pre-mRNA.

In some embodiments, the splice regulation site may regulate splicing of one or more of exon 1, intron 1 and exon 2 of the human progranulin pre-mRNA transcript.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleotide

The term “modified nucleoside” or “nucleoside modification” as used herein refers to nucleosides modified as compared to the equivalent DNA or RNA nucleoside by the introduction of one or more modifications of the sugar moiety or the (nucleo) base moiety.

The antisense oligonucleotide of the invention may comprise a contiguous nucleotide sequence comprising one or more 2′-MOE nucleosides.

The antisense oligonucleotide of the invention may comprise a contiguous nucleotide sequence which comprises one or more further or additionally modified nucleosides which comprise a modified sugar moiety.

The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein.

Nucleosides with modifications in the base region of the DNA or RNA nucleoside are still generally termed DNA or RNA if they allow Watson Crick base pairing.

Exemplary modified nucleosides which may be used in the compounds of the invention include LNA, 2′-O-MOE and morpholino nucleoside analogues.

### Methanesulfonyl Phosphoramidate Internucleotide Linkages

The contiguous nucleotide sequence of the antisense oligonucleotide of the invention comprises one or more modified internucleoside linkages, which are methanesulfonyl phosphoramidate internucleotide linkages.

The methanesulfonyl phosphoramidate internucleoside linkages have one of the non-bridging oxygen atoms in the phosphodiester linkage replaced with a methanesulfonylamido group, with this linkage differing from other phosphoramidate and alkylphosphonate linkages in that it retains negative charge on the phosphate backbone, but lacks a negatively charged sulphur atom that is the primary pharmacophore for ASO-protein interactions.

The methanesulfonyl phosphoramidate internucleoside linkage can also be termed mesyl phosphoramidate, methanesulfonyl phosphoramidate or N-methanesulfonyl phosphoramidate in the literature, where mesyl is short for methanesulfonyl and “N-” specifies the position of the methanesulfonyl on the nitrogen. Herein these terms may be used interchangeably.

In some embodiments at least 50% of the internucleoside linkages in the antisense oligonucleotide, or contiguous nucleotide sequence thereof, are methanesulfonyl, such as at least 60%, such as at least 70%, such as at least 75%, such as at least 80%, such as at least 90%, such as at least 95%, or more of the internucleoside linkages in the antisense oligonucleotide, or contiguous nucleotide sequence thereof, are methanesulfonyl.

In some embodiments, the contiguous nucleotide sequence comprises 1, 2, 3, 4, 5, 6, 7, 8, 9, 10, 11, 12, 13, 14, 15, 16, 17 or more methanesulfonyl phosphoramidate internucleotide linkages.

In some embodiments, the contiguous nucleotide sequence comprises 1 or more methanesulfonyl phosphoramidate internucleotide linkages.

In some embodiments, the contiguous nucleotide sequence comprises 2 or more methanesulfonyl phosphoramidate internucleotide linkages.

In some embodiments, the contiguous nucleotide sequence comprises 3 or more methanesulfonyl phosphoramidate internucleotide linkages.

In some embodiments, the contiguous nucleotide sequence comprises 4 or more methanesulfonyl phosphoramidate internucleotide linkages.

In some embodiments, the contiguous nucleotide sequence comprises 5 or more methanesulfonyl phosphoramidate internucleotide linkages.

In some embodiments, the contiguous nucleotide sequence comprises 6 or more methanesulfonyl phosphoramidate internucleotide linkages.

In some embodiments, the contiguous nucleotide sequence comprises 7 or more methanesulfonyl phosphoramidate internucleotide linkages.

In some embodiments, the contiguous nucleotide sequence comprises 8 or more methanesulfonyl phosphoramidate internucleotide linkages.

In some embodiments, the contiguous nucleotide sequence comprises 9 or more methanesulfonyl phosphoramidate internucleotide linkages.

In some embodiments, the contiguous nucleotide sequence comprises 10 or more methanesulfonyl phosphoramidate internucleotide linkages.

In some embodiments, the contiguous nucleotide sequence comprises 11 or more methanesulfonyl phosphoramidate internucleotide linkages.

In some embodiments, the contiguous nucleotide sequence comprises 12 or more methanesulfonyl phosphoramidate internucleotide linkages.

In some embodiments, the contiguous nucleotide sequence comprises 13 or more methanesulfonyl phosphoramidate internucleotide linkages.

In some embodiments, the contiguous nucleotide sequence comprises 14 or more methanesulfonyl phosphoramidate internucleotide linkages.

In some embodiments, the contiguous nucleotide sequence comprises 15 or more methanesulfonyl phosphoramidate internucleotide linkages.

In some embodiments, the contiguous nucleotide sequence comprises 16 or more methanesulfonyl phosphoramidate internucleotide linkages.

In some embodiments, the contiguous nucleotide sequence comprises 17 or more methanesulfonyl phosphoramidate internucleotide linkages.

Advantageously, all the internucleoside linkages of the contiguous nucleotide sequence of the antisense oligonucleotide may be methanesulfonyl, or all the internucleoside linkages of the antisense oligonucleotide may be methanesulfonyl linkages.

### Modified Internucleoside Linkage

The contiguous nucleotide sequence of the antisense oligonucleotide of the invention may comprise one or more modified internucleoside linkages. It will be apparent to the skilled person that since the contiguous nucleotide sequence of the antisense oligonucleotide of the invention must comprise one or more methanesulfonyl phosphoramidate internucleotide linkages, alternatively modified internucleoside linkages would be additional modified internucleoside linkages.

The term “modified internucleoside linkage” is defined as generally understood by the skilled person as linkages other than phosphodiester (PO) linkages that covalently couple two nucleosides together.

In some embodiments, at least about 75%, at least about 80%, at least about 85%, at least about 90%, at least about 95%, or about 100% of the internucleotide linkages of the contiguous nucleotide sequence are modified.

In some embodiments, all of the internucleotide linkages positioned between the nucleotides of the contiguous nucleotide sequence are modified.

In some embodiments, the contiguous nucleotide sequence comprises one or more phosphorothioate internucleotide linkages.

In some embodiments, the contiguous nucleotide sequence comprises multiple phosphorothioate internucleotide linkages, such as 1, 2, 3, 4, 5, 6, 7, 8, 9, 10 or more phosphorothioate internucleotide linkages.

In some embodiments, all the internucleotide linkages present in the antisense oligonucleotide are selected from phosphorothioate internucleotide linkages and methanesulfonyl phosphoramidate internucleotide linkages.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al. (2012) Accounts of Chemical Research vol. 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The antisense oligonucleotide of the invention may be a modified oligonucleotide.

The term modified oligonucleotide describes an oligonucleotide comprising one or more sugar-modified nucleosides and/or modified internucleoside linkages. The term “chimeric oligonucleotide” is a term that has been used in the literature to describe oligonucleotides comprising sugar modified nucleosides and DNA nucleosides. In some embodiments, it may be advantageous for the antisense oligonucleotide of the invention to be a chimeric oligonucleotide.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U).

It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al. (2012) Accounts of Chemical Research vol. 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

Within the present invention the term “complementary” requires the antisense oligonucleotide to be at least about 80% complementary, or at least about 90% complementary, to a human progranulin pre-mRNA transcript. In some embodiments the antisense oligonucleotide may be at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% complementary to a human progranulin pre-mRNA transcript. Put another way, for some embodiments, an antisense oligonucleotide of the invention may include one, two, three or more mis-matches, wherein a mis-match is a nucleotide within the antisense oligonucleotide of the invention which does not base pair with its target.

The term “fully complementary” refers to 100% complementarity.

The antisense oligonucleotides of the invention are complementary to the human progranulin pre-mRNA. The antisense oligonucleotides of the invention are advantageously complementary to the intron 1 sequence of the human progranulin pre-mRNA transcript. The sequence of exon 1, intron 1 and exon 2 of the human progranulin pre-mRNA transcript is exemplified herein as SEQ ID NO: 1. SEQ ID NO: 1 is provided herein as a reference sequence and it will be understood that the target progranulin nucleic acid may be an allelic variant of SEQ ID NO: 1, such as an allelic variant which comprises one or more polymorphism in the human progranulin nucleic acid sequence.

### Identity

The term “identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif).

The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The terms “hybridizing” or “hybridizes” as used herein are to be understood as two nucleic acid strands (e.g. an antisense oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (Kd) of the reaction by ΔG°=−RTln(Kd), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov. Today. The skilled person will know that commercial equipment is available for ΔG° measurements. ΔG° can also be estimated numerically by using the nearest neighbour model as described by SantaLucia, 1998, Proc. Natl. Acad. Sci. USA. 95:1460-1465 using appropriately derived thermodynamic parameters described by Sugimoto et al., 1995, Biochemistry 34:11211-11216 and McTigue et al., 2004, Biochemistry 43:5388-5405.

In some embodiments, antisense oligonucleotides of the present invention hybridize to a target nucleic acid with estimated ΔG° values below −10 kcal for oligonucleotides that are 10-30 nucleotides in length.

In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal, or −16 to −27 kcal such as −18 to −25 kcal.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (Tm). A high affinity modified nucleoside of the present invention preferably results in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann, 1997, Nucl. Acid Res., 25, 4429-4443 and Uhlmann, 2000, Curr. Opinion in Drug Development, 3 (2), 293-213).

### Sugar Modifications

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or −OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been given to developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide.

Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann, 1997, Nucl. Acid Res., 25, 4429-4443 and Uhlmann, 2000, Curr. Opinion in Drug Development, 3 (2), 293-213, and Deleavey and Damha, 2012, Chemistry and Biology, 19, 937. Below are illustrations of some 2′ substituted modified nucleosides.

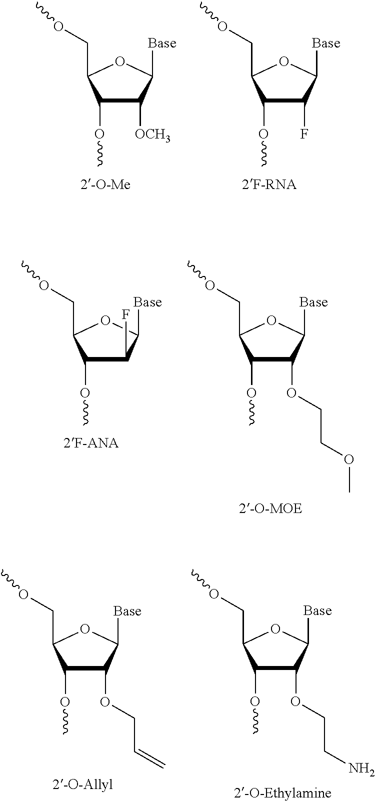

In relation to the present invention 2′-substituted sugar modified nucleosides do not include 2′ bridged nucleosides like LNA.

### Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al., 2010, J. Org. Chem., Vol 75 (5) pp. 1569-81, and Mitsuoka et al., 2009, Nucleic Acids Research, 37 (4), 1225-1238, and Wan and Seth, 2016, J. Medical Chemistry, 59, 9645-9667.

Further non limiting, exemplary LNA nucleosides are disclosed in Scheme 1.

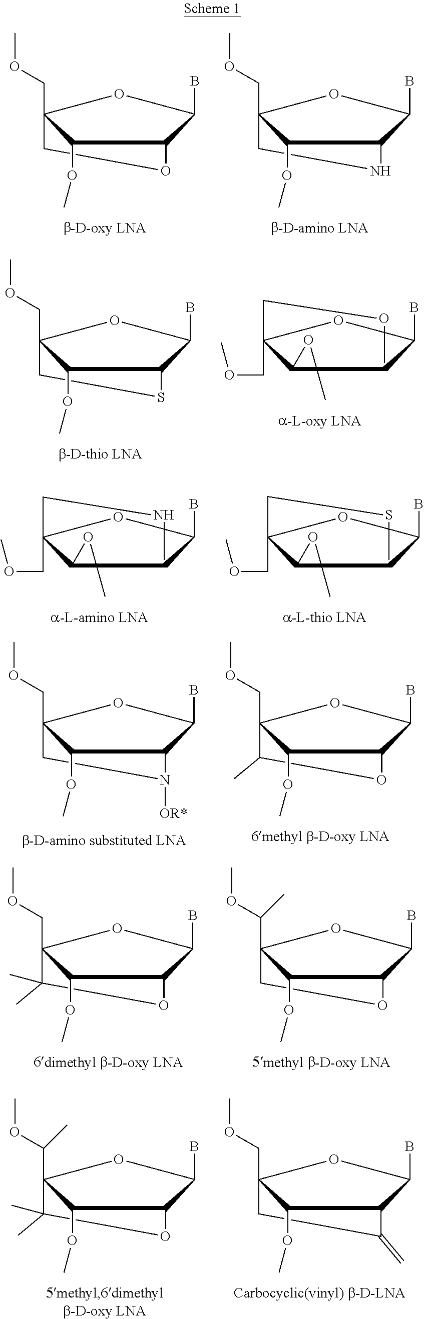

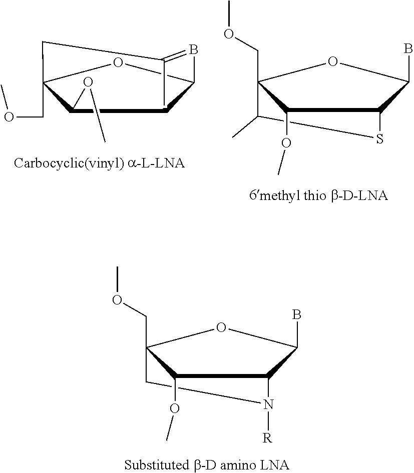

Particular LNA nucleosides are beta-D-oxy-LNA, 6′-methyl-beta-D-oxy LNA such as(S)-6′-methyl-beta-D-oxy-LNA (ScET) and ENA.

A particularly advantageous LNA is beta-D-oxy-LNA.

### Morpholino Oligonucleotides

In some embodiments, the antisense oligonucleotide of the invention comprises or consists of morpholino nucleosides (i.e. is a Morpholino oligomer and as a phosphorodiamidate Morpholino oligomer (PMO)). Splice modulating morpholino oligonucleotides have been approved for clinical use-see for example eteplirsen, a 30 nt morpholino oligonucleotide targeting a frame shift mutation in DMD, used to treat Duchenne muscular dystrophy.

Morpholino oligonucleotides have nucleobases attached to six membered morpholine rings rather ribose, such as methylenemorpholine rings linked through phosphorodiamidate groups, for example as illustrated by the following illustration of 4 consecutive morpholino nucleotides:

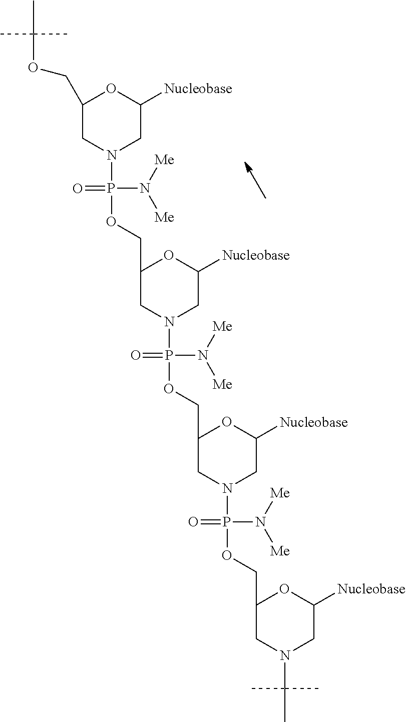

In some embodiments, morpholino oligonucleotides of the invention may be, for example 20-40 morpholino nucleotides in length, such as morpholino 25-35 nucleotides in length.

### Rnase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10%, at least 20% or more than 20%, of the initial rate determined when using an oligonucleotide having the same base sequence as the modified oligonucleotide being tested, but containing only DNA monomers with phosphorothioate linkages between all monomers in the oligonucleotide, and using the methodology provided by Examples 91-95 of WO01/23613 (hereby incorporated by reference). For use in determining RHase H activity, recombinant RNase H1 is available from Lubio Science GmbH, Lucerne, Switzerland.

DNA oligonucleotides are known to effectively recruit RNaseH, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA. For effective modulation of splicing, degradation of the pre-mRNA is not desirable, and as such it is preferable to avoid the RNaseH degradation of the target. Therefore, the antisense oligonucleotides of the invention are not RNaseH recruiting gapmer oligonucleotide.

RNaseH recruitment may be avoided by limiting the number of contiguous DNA nucleotides in the oligonucleotide—therefore totalmer designs may be used.

### Mixmers and Totalmers

For splice modulation it is often advantageous to use antisense oligonucleotides which do not recruit RNAaseH. As RNaseH activity requires a contiguous sequence of DNA nucleotides, RNaseH activity of antisense oligonucleotides may be achieved by designing antisense oligonucleotides which do not comprise a region of more than 3 or more than 4 contiguous DNA nucleosides. This may be achieved by using antisense oligonucleotides or contiguous nucleoside regions thereof with a mixmer design, which comprise sugar modified nucleosides, such as 2′ sugar modified nucleosides, and short regions of DNA nucleosides, such as 1, 2 or 3 DNA nucleosides. Mixmers are exemplified herein by every second design, wherein the nucleosides alternate between 1 LNA and 1 DNA nucleoside, e.g. LDLDLDLDLDLDLDLL, with 5′ and 3′ terminal LNA nucleosides, and every third design, such as LDDLDDLDDLDDLDDL, where every third nucleoside is a LNA nucleoside.

A totalmer is an antisense oligonucleotide or a contiguous nucleotide sequence thereof which does not comprise DNA or RNA nucleosides, and may for example comprise only 2′-O-MOE nucleosides, such as a fully MOE phosphorothioate, e.g. MMMMMMMMMMMMMMMMMMMM, where M=2′-O-MOE, which are reported to be effective splice modulators for therapeutic use.

Alternatively, a mixmer may comprise a mixture of modified nucleosides, such as MLMLMLMLMLMLMLMLMLML, wherein L=LNA and M=2′-O-MOE nucleosides. Advantageously, the internucleoside nucleosides in mixmers and totalmers may include one or more phosphorothioate internucleotide linkages and one or more methanesulfonyl phosphoramidate internucleotide linkages, or a majority of nucleoside linkages in mixmers may be phosphorothioate internucleotide linkages and methanesulfonyl phosphoramidate internucleotide linkages. Mixmers and totalmers may comprise other internucleoside linkages, such as phosphodiester or phosphorodithioate, by way of example.

### Region D′ or D″ in an Oligonucleotide

The antisense oligonucleotide of the invention may in some embodiments comprise or consist of the contiguous nucleotide sequence of the oligonucleotide which is complementary to the target nucleic acid, such as totalmer region, and further 5′ and/or 3′ nucleosides. The further 5′ and/or 3′ nucleosides may or may not be complementary, such as fully complementary, to the target nucleic acid. Such further 5′ and/or 3′ nucleosides may be referred to as region D′ and D″ herein.

The addition of region D′ or D″ may be used for the purpose of joining the contiguous nucleotide sequence, such as the totalmer, to a conjugate moiety or another functional group. When used for joining the contiguous nucleotide sequence with a conjugate moiety it can serve as a biocleavable linker. Alternatively, it may be used to provide exonuclease protection or for ease of synthesis or manufacture.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D″ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In one embodiment the antisense oligonucleotide of the invention comprises a region D′ and/or D″ in addition to the contiguous nucleotide sequence which constitutes a totalmer.

In some embodiments the internucleoside linkage positioned between region D′ or D″ and the totalmer region is a phosphodiester linkage.

### Conjugate

The invention encompasses an antisense oligonucleotide covalently attached to at least one conjugate moiety. In some embodiments this may be referred to as a conjugate of the invention.

The term “conjugate” as used herein refers to an antisense oligonucleotide which is covalently linked to a non-nucleotide moiety (conjugate moiety or region C or third region).

The conjugate moiety may be covalently linked to the antisense oligonucleotide, optionally via a linker group, such as region D′ or D″.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, 2002, Antisense and Nucleic Acid Drug Development, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the antisense oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or antisense oligonucleotide conjugate of the invention may optionally comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The antisense oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In some embodiments the linker (region Y) is a C6 amino alkyl group.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### TDP-43 Pathologies

A TDP-43 pathology is a disease which is associated with reduced or aberrant expression of TDP-43, often associated with an increase in cytoplasmic TDP-43, particularly hyper-phosphorylated and ubiquitinated TDP-43.

Diseases associated with TDP-43 pathology include amyotrophic lateral sclerosis (ALS), frontotemporal lobar degeneration (FTLD), Alzheimer's disease, Parkinson's disease, Autism, Hippocampal sclerosis dementia, Down syndrome, Huntington's disease, polyglutamine diseases, such as spinocerebellar ataxia 3, myopathies and Chronic Traumatic Encephalopathy.

### DETAILED DESCRIPTION OF THE INVENTION

The inventors have identified that targeting the progranulin pre-mRNA transcript with antisense oligonucleotides can increase expression of the progranulin Exon1-Exon 2 spliced mRNA, decrease expression of the progranulin Intron1-Exon2 spliced mRNA (which retains the 271 nucleotide 5′ fragment of intron 1) and/or alter the ratio of Exon1-Exon2 vs Intron1-Exon2 mRNA. This is particularly the case when antisense oligonucleotides which comprise one or more methanesulfonyl phosphoramidate internucleotide linkages are used.

Described herein are target sites present on the human progranulin pre-mRNA which can be targeted by antisense oligonucleotides. Also described are antisense oligonucleotides which are complementary, such as fully complementary, to these target sites.

Without wishing to be bound by theory, it is considered that the antisense oligonucleotides of the invention can increase expression of the progranulin Exon1-Exon2 spliced mRNA, decrease expression of the progranulin Intron1-Exon1 spliced mRNA and/or alter the ratio of Exon1-Exon2 vs Intron1-Exon2 mRNA by binding to these regions and affecting, such as increasing, production of the Exon1-Exon2 splice variant.

Oligonucleotides, such as RNaseH recruiting single stranded antisense oligonucleotides or siRNAs are used extensively in the art to inhibit target RNAs—i.e. are used as antagonists of their complementary nucleic acid target.

The antisense oligonucleotides of the present invention may be described as modulators, i.e. they alter the expression of a particular splice variant of their complementary target, progranulin pre-mRNA, and thereby increase the production of active progranulin protein.

Reduced expression of the progranulin Intron1-Exon2 splice variant is desirable because the inclusion of an intron, such as Intron 1, within a mature mRNA sequence leads to nonsense-mediated mRNA decay (NMD).

Enhanced expression of the progranulin Exon1-Exon2 over the splice variant which retains the 5′ part of intron 1 is desirable because the Exon1-Exon2 splice variant does not include the 271 nucleotide fragment of intron 1 with two AUG sites upstream of the canonical downstream AUG in Exon 2 (open reading frame). Translation from these two upstream AUG sites will not encode the progranulin protein and due to premature termination codons the transcript may undergo non-sense mediated mRNA decay (NMD). Changing the splicing to the Exon1-Exon2 splice variant will instead lead to translation of an active version of the progranulin protein. Progranulin is a neuroprotective protein, and increasing its production can be used to treat a range of neurological disorders, such as TDP-43 pathologies.

In certain embodiments the antisense oligonucleotides of the present invention may enhance the production of the Exon1-Exon2 progranulin splice variant.

In certain embodiments the antisense oligonucleotides of the present invention may enhance the production of the Exon1-Exon2 progranulin splice variant mRNA by at least about 10% relative to the production of the Exon1-Exon2 progranulin splice variant mRNA in the absence of an antisense oligonucleotide of the invention. In other embodiments the antisense oligonucleotides of the present invention may enhance the production of the Exon1-Exon2 progranulin splice variant mRNA by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500% or more relative to the production of the Exon1-Exon2 progranulin splice variant mRNA in the absence of an antisense oligonucleotide of the invention.

In certain embodiments the antisense oligonucleotides of the present invention may reduce the production of the Intron1-Exon2 progranulin splice variant mRNA.

In certain embodiments the antisense oligonucleotides of the present invention may reduce the production of the Intron1-Exon2 progranulin splice variant mRNA by at least about 10% relative to the production of the Intron1-Exon2 progranulin splice variant mRNA in the absence of an antisense oligonucleotide of the invention. In other embodiments the antisense oligonucleotides of the present invention may reduce the production of the Intron1-Exon2 progranulin splice variant mRNA by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500% or more relative to the production of the Intron1-Exon2 progranulin splice variant mRNA in the absence of an antisense oligonucleotide of the invention.

Enhanced expression of the progranulin Exon1-Exon2 splice variant should lead to translation of an active version of the progranulin protein. In certain embodiments the antisense oligonucleotides of the present invention may increase production of the progranulin protein by at least about 10% relative to the production of the progranulin protein in the absence of an antisense oligonucleotide of the invention. In other embodiments the antisense oligonucleotides of the present invention may increase the production of the progranulin protein by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500% or more relative to the production of the progranulin protein in the absence of an antisense oligonucleotide of the invention.

In certain embodiments, the antisense oligonucleotides of the present invention may alter the ratio of Exon 1-Exon2 vs Intron-Exon2 progranulin mRNA.

In certain embodiments, the antisense oligonucleotides of the present invention may alter the ratio of Exon1-Exon2 vs Intron1-Exon2 progranulin mRNA by at least about 10% relative to the ratio of Exon1-Exon2 vs Intron1-Exon2 progranulin mRNA in the absence of an antisense oligonucleotide of the invention. In other embodiments the antisense oligonucleotides of the present invention may alter the ratio of Exon1-Exon2 vs Intron1-Exon2 progranulin mRNA by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, relative to the ratio of Exon1-Exon2 vs Intron1-Exon2 progranulin mRNA in the absence of an antisense oligonucleotide of the invention.

In certain embodiments, the antisense oligonucleotides of the present invention may alter the ratio of Exon1-Exon2 vs Intron1-Exon2 progranulin mRNA to at least about 1.2. In certain embodiments, the antisense oligonucleotides of the present invention may alter the ratio of Exon1-Exon2 vs Intron1-Exon2 progranulin mRNA to at least about 1.3, at least about 1.4, at least about 1.5, at least about 1.6, at least about 1.7, at least about 1.8, at least about 1.9, at least about 2.0 or more.

In some embodiments, the antisense oligonucleotides of the invention or the contiguous nucleotide sequence thereof comprises or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 contiguous nucleotides in length.

In some embodiments, the entire nucleotide sequence of the antisense oligonucleotide is the contiguous nucleotide sequence.

In one embodiment the contiguous nucleotide sequence may be a sequence selected from the group consisting of SEQ ID NO: 5, SEQ ID NO: 6, SEQ ID NO: 7, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 30, SEQ ID NO: 31, SEQ ID NO: 34, SEQ ID NO: 35, SEQ ID NO: 36 and SEQ ID NO: 37. The invention also contemplates fragments of these contiguous nucleotide sequences, including fragments of at least 8, at least 9, at least 10, at least 11, at least 12, at least 13, at least 14, or at least 15 contiguous nucleotides thereof.

In some embodiments, the antisense oligonucleotide or contiguous nucleotide sequence comprises or consists of a sequence selected from the group consisting of SEQ ID NO: 5, SEQ ID NO: 6, SEQ ID NO: 7, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 30, SEQ ID NO: 31, SEQ ID NO: 34, SEQ ID NO: 35, SEQ ID NO: 36 and SEQ ID NO: 37.

It will be understood that the sequences shown in SEQ ID NO: 5, SEQ ID NO: 6, SEQ ID NO: 7, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 30, SEQ ID NO: 31, SEQ ID NO: 34, SEQ ID NO: 35, SEQ ID NO: 36 and SEQ ID NO: 37 may include modified nucleobases which function as the shown nucleobase in base pairing, for example 5-methyl cytosine may be used in place of methyl cytosine. Inosine may be used as a universal base.

In some embodiments, the antisense oligonucleotide or contiguous nucleotide sequence comprises or consists of 8 to 30 or 8 to 40 nucleotides in length with at least 90% identity, preferably 100% identity, to a sequence selected from the group consisting of SEQ ID NO: 5, SEQ ID NO: 6, SEQ ID NO: 7, SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 27, SEQ ID NO: 28, SEQ ID NO: 30, SEQ ID NO: 31, SEQ ID NO: 34, SEQ ID NO: 35, SEQ ID NO: 36 and SEQ ID NO: 37.

In some embodiments the antisense oligonucleotide may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

It is understood that the contiguous nucleobase sequences (motif sequence) can be modified to, for example, increase nuclease resistance and/or binding affinity to the target nucleic acid.

The pattern in which the modified nucleosides (such as high affinity modified nucleosides) are incorporated into the oligonucleotide sequence is generally termed oligonucleotide design.

The antisense oligonucleotides of the invention are designed with modified nucleosides and DNA nucleosides. Advantageously, high affinity modified nucleosides are used.

In an embodiment, the antisense oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides.

In an embodiment the antisense oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the antisense oligonucleotide comprises one or more sugar modified nucleosides, such as 2′ sugar modified nucleosides. Preferably the antisense oligonucleotide of the invention comprises one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

### EXAMPLES

### Example 1

hiPSC-derived micoglia (iCell Microglia Kit, 01279, Cat. no R1131) were seeded (n=1) in Poly-D-lysine coated 96-well plates (Greiner Catalog #655946) with 20000 cells per well in 200 μL and were treated with indicated concentrations of 3 UM and 10 UM of SEQ ID NOs 2-38 for 5 days.

RNA was extracted by adding 125 μL RLT buffer (Qiagen) and using RNeasy 97 kit and protocols from Qiagen. cDNA synthesis was performed using 4 μL input RNA with IScript Advanced cDNA Synthesis Kit for RT-qPCR (Bio-Rad) and 2 μL RNA was used as input for digital droplet PCR using ddPCR supermix for probes (no dUTP) (Bio-Rad) according to the Manufacturer's protocol. The following Primers and Probes (IDT) were used:

GRN Exon1-Exon2 (FAM):
Primer 1:
(SEQ ID NO: 40)
GCTGCTGCCCAAGGACCGCGGA
Primer 2:
(SEQ ID NO: 41)
GCCCTGCTGTTAAGGCCACCCA
Probe
(SEQ ID NO: 42)
/56-FAM/GGACGCAGG/ZEN/CAGACCATGTGGACCCTG/3IABkFQ/
GRN Intron1-Exon2 (HEX):
Primer 1:
(SEQ ID NO: 43)
CCAAAGCAGGGACCACACCATTCTT
Primer 2:
(SEQ ID NO: 44)
GCCCTGCTGTTAAGGCCACCCA
Probe
(SEQ ID NO: 45)
/5HEX/CCCAGCTCC/ZEN/ACCCCTGTCGGCAGACCATG/3IABkFQ/
GAPDH:
GAPDH (FAM, Hs.PT.39a.22214836, IDT)
GAPDH (HEX, Hs.PT.39a.22214836, IDT)

Exon1-Exon2 GRN mRNA and Intron1-Exon2 GRN mRNA concentrations were quantified relative to the housekeeping gene GAPDH using QuantaSoft Software (Bio-Rad).

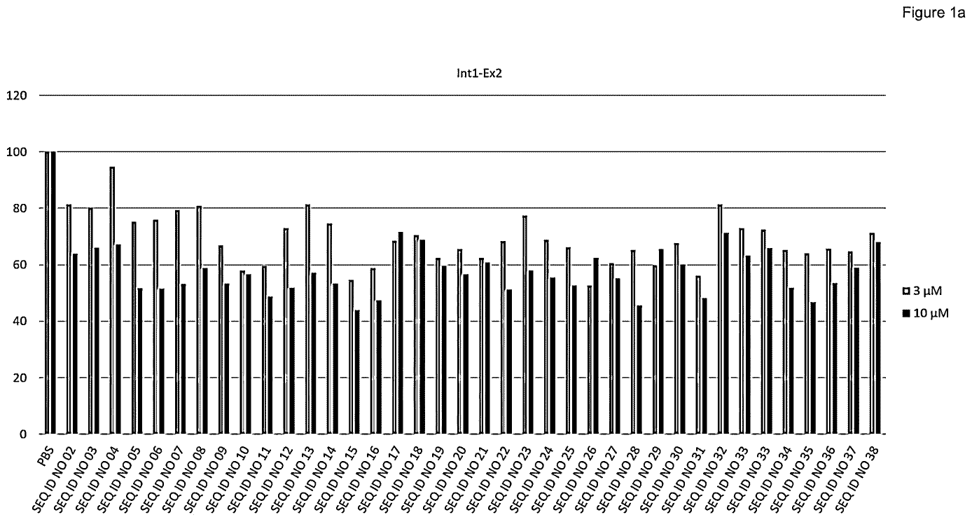

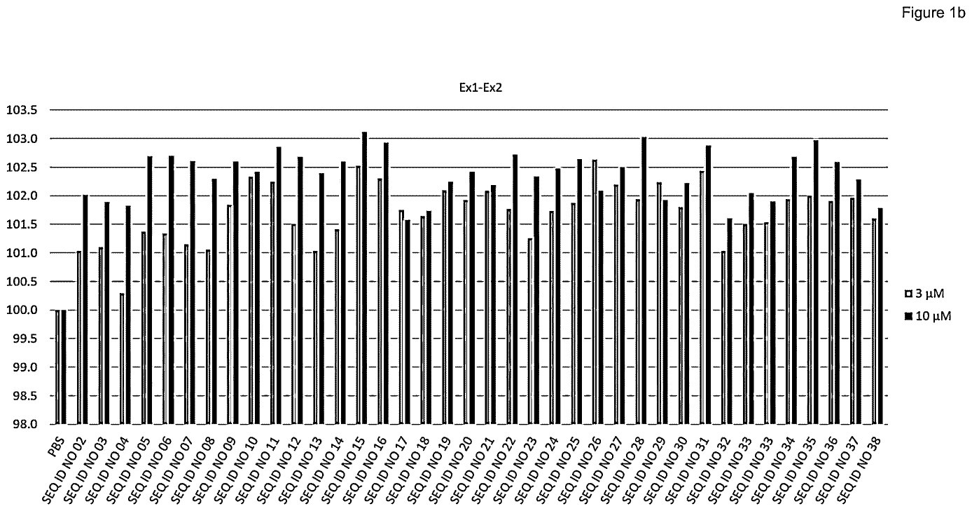

The results are shown in FIGS. 1a & 1b. Compared to SEQ ID NO: 2, SEQ ID NOs 5, 6, 7, 9, 10, 11, 12, 14, 15, 16, 19, 20, 21, 22, 23, 24, 25, 27, 28, 30, 31, 34, 35, 36 and 37 show efficient and improved skipping of intron1 retention (Int1-Ex2) and a potential minor increase in Exon1-Exon2 (Ex1-Ex2) splice-variant at both 3 UM and 10 μM doses. SEQ ID NO: 33 was tested twice and so has duplicate results in both FIGS. 1a & 1b.

### Compound Table

### HELM Notation

Antisense oligonucleotides (compounds) of the invention and antisense oligonucleotide conjugates (conjugates) of the invention are depicted herein using Hierarchical Editing Language for Macromolecules (HELM) notation.

HELM is a notation format designed to depict the structure of macromolecules. Full details of HELM notation may be found at www.pistoiaalliance.org/helm-tools/, in Zhang et al., 2012, J. Chem. Inf. Model., 52, 2796-2806 (which initially described HELM notation) and in Milton et al., 2017, J. Chem Inf. Model., 57, 1233-1239 (which describes HELM version 2.0).

Briefly, a macromolecule is depicted as a “HELM string”, which is divided into sections. The first section lists the molecules comprised in the macromolecule. The second section lists the connections between molecules within the macromolecule. One or more dollar sign $ marks the end of a section of a HELM string.

Compounds of the invention are represented by a HELM string consisting of a single first section defining the oligonucleotide.

Each molecule listed in the first section of a HELM string is given an identifier (e.g. “RNA1” for a nucleic acid) and the structure of the molecule is defined by notation in braces { } immediately following the identifier. The HELM notations used to define the structure of each molecule in braces { } in the first section of HELM strings for the compounds and conjugates of the present invention are as follows:
[MOE](G) is a 2′-O-(2-methoxy) ethyl RNA guanine nucleoside
[MOE](U) is a 2′-O-(2-methoxy) ethyl RNA uracil nucleoside
[MOE](A) is a 2′-O-(2-methoxy) ethyl RNA adenine nucleoside
[MOE]([5meC]) is a 2′-O-(2-methoxy) ethyl RNA 5-methyl cytosine nucleoside
MOE](T) is a 2′-O-(2-methoxy) ethyl thymine nucleoside
[sP] is a phosphorothioate backbone
[MsNP] is a methanesulfonyl phosphoramidate backbone

As noted above, in the context of the invention, a second section is used only in HELM strings representing conjugates of the invention. This second section lists the connections between the molecules listed in the first section. Each pair of molecules that are connected are defined by listing their identifiers, and then the attachment points between them (i.e. the point at which there is a covalent bond between the molecules) are defined.

### Examples of HELM Notation

For example, SEQ ID NO: 2 is represented by the following HELM string (as depicted in the Compound Table of Example 1):

RNA1{[MOE](G)[sP].[MOE](G)[sP].[MOE](T)[sP].[MOE]
([5meC])[sP].[MOE](A)[sP].[MOE](A)[sP].[MOE](G)
[sP].[MOE](A)[sP].[MOE](A)[sP].[MOE](T)[sP].[MOE]
(G)[sP].[MOE](G)[sP].[MOE](T)[sP].[MOE](G)[sP].
[MOE](T)[sP].[MOE](G)[sP].[MOE](G)[sP].[MOE]
(T)}$$$$V2.0

This HELM string consists of a single section listing the oligonucleotide of SEQ ID NO 2. The initial “RNA1” indicates the molecule is a nucleic acid (oligonucleotide). The structure of the oligonucleotide is presented using HELM notation in the braces { } following RNA1. “$$$$” marks the end of the section, and of the HELM string as a whole. “V2.0” indicates that HELM version 2.0 is used.

### HELM Annotation Key:

[MOE](G) is a 2′-O-(2-methoxy) ethyl RNA guanine nucleoside
[MOE](U) is a 2′-O-(2-methoxy) ethyl RNA uracil nucleoside
[MOE](A) is a 2′-O-(2-methoxy) ethyl RNA adenine nucleoside
[MOE]([5meC]) is a 2′-O-(2-methoxy) ethyl RNA 5-methyl cytosine nucleoside
MOE](T) is a 2′-O-(2-methoxy) ethyl thymine nucleoside
[sP] is a phosphorothioate backbone
[MsNP] is a methanesulfonyl phosphoramidate backbone

| | | | | | Start | End | | |
|---|---|---|---|---|---|---|---|---|
| | | | | Target | position in | position in | Chromosome | Chromosome |
| SEQ | Oligo | | Target | SEQ ID | SEQ ID | SEQ ID | start (hg38 | end (hg38 |
| ID NO | Sequence | HELM sequence | sequence | NO | NO: 1 | NO: 1 | assembly) | assembly) |
| 2 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[P].[MOE](G)[sP].[MOE] | | | | | | |
| | | (G)[sP].[MOE](T)[sP].[MOE](G)[sP]. | | | | | | |
| | | [MOE](T)[sP].[MOE](G)[sP].[MOE] | | | | | | |
| | | (G)[sP].[MOE](T)}$$$$V2.0 | | | | | | |
| 3 | GGTCAAG | RNA1{[MOE](G)[MsNP].[MOE](G) | ACCACA | 39 | 449 | 466 | 44345571 | 44345588 |
| | AATGGTG | [MsNP].[MOE](T)[sP].[MOE]([5meC]) | CCATTCT | | | | | |
| | TGGT | [sP].[MOE](A)[sP].[MOE](A)[sP]. | TGACC | | | | | |
| | | [MOE](G)[sP].[MOE](A)[sP].[MOE] | | | | | | |
| | | (A)[sP].[MOE](T)[sP].[MOE](G)[sP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 4 | GGTCAAG | RNA1{[MOE](G)[sP].[MOE](G)[MsNP]. | ACCACA | 39 | 449 | 466 | 44345571 | 44345588 |
| | AATGGTG | [MOE](T)[MsNP].[MOE]([5meC]) | CCATTCT | | | | | |
| | TGGT | [sP].[MOE](A)[sP].[MOE](A)[sP]. | TGACC | | | | | |
| | | [MOE](G)[sP].[MOE](A)[sP].[MOE] | | | | | | |
| | | (A)[sP].[MOE](T)[sP].[MOE](G[sP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 5 | GGTCAAG | RNA1{[MOE](G)[sP].[MOE](G)[sP]. | ACCACA | 39 | 449 | 466 | 44345571 | 44345588 |
| | AATGGTG | [MOE](T)[MsNP].[MOE]([5meC])[MsNP]. | CCATTCT | | | | | |
| | TGGT | [MOE](A)[sP].[MOE](A)[sP]. | TGACC | | | | | |
| | | [MOE](G)[sP].[MOE](A)[sP].[MOE] | | | | | | |
| | | (A)[sP].[MOE](T)[sP].[MOE](G)[sP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 6 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[MsNP]. | CCATTCT | | | | | |
| | TGGT | [MOE](A)[MsNP].[MOE](A)[sP]. | TGACC | | | | | |
| | | [MOE](G)[sP].[MOE](A)[sP].[MOE] | | | | | | |
| | | (A)[sP].[MOE](T)[sP].[MOE](G)[sP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 7 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[MsNP].[MOE](A)[MsNP]. | TGACC | | | | | |
| | | [MOE](G)[sP].[MOE](A)[sP].[MOE] | | | | | | |
| | | (A)[sP].[MOE](T)[sP].[MOE](G)[sP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 8 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[MsNP].[MOE] | TGACC | | | | | |
| | | (G)[MsNP].[MOE](A)[sP].[MOE] | | | | | | |
| | | (A)[sP].[MOE](T)[sP].[MOE](G)[sP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 9 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[MsNP].[MOE](A)[MsNP].[MOE] | | | | | | |
| | | (A)[sP].[MOE](T)[sP].[MOE](G)[sP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 10 | GGTCAAG | RNA1{[MOE](G)[s].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[MsNP].[MOE](A) | | | | | | |
| | | [MsNP].[MOE](T)[sP].[MOE](G)[sP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 11 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[MsNP].[MOE](G)[MsNP]. | | | | | | |
| | | [MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 12 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[MsNP].[MOE] | | | | | | |
| | | (G)[MsNP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 13 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[sP].[MOE] | | | | | | |
| | | (G)[MsNP].[MOE](T)[MsNP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 14 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[sP].[MOE] | | | | | | |
| | | (G)[sP].[MOE](T)[sP].[MOE](G)[MsNP]. | | | | | | |
| | | [MOE](T)[MsNP].[MOE](G)[sP] | | | | | | |
| | | .[MOE](G)[sP].[MOE](T)}$$$$V2.0 | | | | | | |
| 15 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[sP].[MOE] | | | | | | |
| | | (G)[sP].[MOE](T)[sP].[MOE](G)[sP]. | | | | | | |
| | | [MOE](T)[MsNP].[MOE](G)[MsNP]. | | | | | | |
| | | [MOE](G)[sP].[MOE](T)}$$$$V2.0 | | | | | | |
| 16 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[sP].[MOE] | | | | | | |
| | | (G)[sP].[MOE](T)[sP].[MOE](G)[sP]. | | | | | | |
| | | [MOE](T)[sP].[MOE](G)[MsNP].[MOE] | | | | | | |
| | | (G)[MsNP].[MOE](T)}$$$$V2.0 | | | | | | |
| 17 | GGTCAAG | RNA1{[MOE](G)[MsNP].[MOE](G) | ACCACA | 39 | 449 | 466 | 44345571 | 44345588 |
| | AATGGTG | [MsNP].[MOE](T)[MsNP].[MOE] | CCATTCT | | | | | |
| | TGGT | ([5meC])[sP].[MOE](A)[sP].[MOE](A) | TGACC | | | | | |
| | | [sP].[MOE](G)[sP].[MOE](A)[sP]. | | | | | | |
| | | [MOE](A)[sP].[MOE](T)[sP].[MOE](G) | | | | | | |
| | | [sP].[MOE](G)[sP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V | | | | | | |
| | | 2.0 | | | | | | |
| 18 | GGTCAAG | RNA1{[MOE](G)[sP].[MOE](G)[MsNP]. | ACCACA | 39 | 449 | 466 | 44345571 | 44345588 |
| | AATGGTG | [MOE](T)[MsNP].[MOE]([5meC]) | CCATTCT | | | | | |
| | TGGT | [MsNP].[MOE](A)[sP].[MOE](A) | TGACC | | | | | |
| | | [sP].[MOE](G)[sP].[MOE](A)[sP]. | | | | | | |
| | | [MOE](A)[sP].[MOE](T)[sP].[MOE](G) | | | | | | |
| | | [sP].[MOE](G)[sP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V | | | | | | |
| | | 2.0 | | | | | | |
| 19 | GGTCAAG | RNA1{[MOE](G)[sP].[MOE](G)[sP]. | ACCACA | 39 | 449 | 466 | 44345571 | 44345588 |
| | AATGGTG | [MOE](T)[MsNP].[MOE]([5meC]) | CCATTCT | | | | | |
| | TGGT | [MsNP].[MOE](A)[MsNP].[MOE](A) | TGACC | | | | | |
| | | [sP].[MOE](G)[sP].[MOE](A)[sP]. | | | | | | |
| | | [MOE](A)[sP].[MOE](T)[sP].[MOE](G) | | | | | | |
| | | [sP].[MOE](G)[sP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V2. | | | | | | |
| | | 0 | | | | | | |
| 20 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[MsNP]. | CCATTCT | | | | | |
| | TGGT | [MOE](A)[MsNP].[MOE](A)[MsNP]. | TGACC | | | | | |
| | | [MOE](G)[sP].[MOE](A)[sP].[MOE] | | | | | | |
| | | (A)[sP].[MOE](T)[sP].[MOE](G)[sP] | | | | | | |
| | | .[MOE](G)[sP].[MOE](T)[sP].[MOE] | | | | | | |
| | | (G)[sP].[MOE](T)[sP].[MOE](G)[sP]. | | | | | | |
| | | [MOE](G)[sP].[MOE](T)}$$$$V2.0 | | | | | | |
| 21 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[MsNP].[MOE](A)[MsNP]. | TGACC | | | | | |
| | | [MOE](G)[MsNP].[MOE](A)[sP]. | | | | | | |
| | | [MOE](A)[sP].[MOE](T)[sP].[MOE](G) | | | | | | |
| | | [sP].[MOE](G)[sP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V | | | | | | |
| | | 2.0 | | | | | | |
| 22 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[MsNP]. | TGACC | | | | | |
| | | [MOE](G)[MsNP].[MOE](A)[MsNP]. | | | | | | |
| | | [MOE](A)[sP].[MOE](T)[sP].[MOE](G) | | | | | | |
| | | [sP].[MOE](G)[sP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V | | | | | | |
| | | 2.0 | | | | | | |
| 23 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[MsNP].[MOE](A)[MsNP].[MOE] | | | | | | |
| | | (A)[MsNP].[MOE](T)[sP].[MOE](G) | | | | | | |
| | | [sP].[MOE](G)[sP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V | | | | | | |
| | | 2.0 | | | | | | |
| 24 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[MsNP].[MOE](A) | | | | | | |
| | | [MsNP].[MOE](T)[MsNP].[MOE](G) | | | | | | |
| | | [sP].[MOE](G)[sP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V | | | | | | |
| | | 2.0 | | | | | | |
| 25 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A) | | | | | | |
| | | [MsNP].[MOE](T)[MsNP].[MOE](G) | | | | | | |
| | | [MsNP].[MOE](G)[sP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V | | | | | | |
| | | 2.0 | | | | | | |
| 26 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[MsNP].[MOE](G)[MsNP]. | | | | | | |
| | | [MOE](G)[MsNP].[MOE](T)[sP]. | | | | | | |
| | | [MOE](G)[sP].[MOE](T)[sP].[MOE](G) | | | | | | |
| | | [sP].[MOE](G)[sP].[MOE](T)}$$$$V2. | | | | | | |
| | | 0 | | | | | | |
| 27 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[MsNP]. | | | | | | |
| | | [MOE](G)[MsNP].[MOE](T)[MsNP]. | | | | | | |
| | | [MOE](G)[sP].[MOE](T)[sP].[MOE](G) | | | | | | |
| | | [sP].[MOE](G)[sP].[MOE](T)}$$$$V2. | | | | | | |
| | | 0 | | | | | | |
| 28 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[sP].[MOE] | | | | | | |
| | | (G)[MsNP].[MOE](T)[MsNP].[MOE] | | | | | | |
| | | (G)[MsNP].[MOE](T)[sP].[MOE](G) | | | | | | |
| | | [sP].[MOE](G)[sP].[MOE](T)}$$$$V | | | | | | |
| | | 2.0 | | | | | | |
| 29 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[P]. | | | | | | |
| | | [MOE](T)[sP].[MOE](G)[sP].[MOE] | | | | | | |
| | | (G)[sP].[MOE](T)[MsNP].[MOE](G) | | | | | | |
| | | [MsNP].[MOE](T)[MsNP].[MOE](G) | | | | | | |
| | | [sP].[MOE](G)[sP].[MOE](T)}$$$$V | | | | | | |
| | | 2.0 | | | | | | |
| 30 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[sP].[MOE] | | | | | | |
| | | (G)[sP].[MOE](T)[sP].[MOE](G) | | | | | | |
| | | [MsNP].[MOE](T)[MsNP].[MOE](G) | | | | | | |
| | | [MsNP].[MOE](G)[sP].[MOE](T)}$$$$V | | | | | | |
| | | 2.0 | | | | | | |
| 31 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[sP].[MOE](A)[sP].[MOE](A)[sP]. | | | | | | |
| | | [MOE](T)[sP].[MOE](G)[sP].[MOE] | | | | | | |
| | | (G)[sP].[MOE](T)[sP].[MOE](G)[sP]. | | | | | | |
| | | [MOE](T)[MsNP].[MOE](G)[MsNP]. | | | | | | |
| | | [MOE](G)[MsNP].[MOE](T)}$$$$V2 | | | | | | |
| | | .0 | | | | | | |
| 32 | GGTCAAG | RNA1{[MOE](G)[MsNP].[MOE](G) | ACCACA | 39 | 449 | 466 | 44345571 | 44345588 |
| | AATGGTG | [MsNP].[MOE](T)[MsNP].[MOE] | CCATTCT | | | | | |
| | TGGT | ([5meC])[MsNP].[MOE](A)[sP].[MOE] | TGACC | | | | | |
| | | (A)[sP].[MOE](G)[sP].[MOE](A)[sP]. | | | | | | |
| | | [MOE](A)[sP].[MOE](T)[sP].[MOE] | | | | | | |
| | | (G)[sP].[MOE](G)[sP].[MOE](T)[sP]. | | | | | | |
| | | [MOE](G)[sP].[MOE](T)[sP].[MOE] | | | | | | |
| | | (G)[sP].[MOE](G)[sP].[MOE](T)}$$$ | | | | | | |
| | | $V2.0 | | | | | | |
| 33 | GGTCAAG | RNA1{[MOE](G)[sP].[MOE](G)[MsNP]. | ACCACA | 39 | 449 | 466 | 44345571 | 44345588 |
| | AATGGTG | [MOE](T)[MsNP].[MOE]([5meC]) | CCATTCT | | | | | |
| | TGGT | [MsNP].[MOE](A)[MsNP].[MOE] | TGACC | | | | | |
| | | (A)[sP].[MOE](G)[sP].[MOE](A)[sP]. | | | | | | |
| | | [MOE](A)[sP].[MOE](T)[sP].[MOE] | | | | | | |
| | | (G)[sP].[MOE](G)[sP].[MOE](T)[sP]. | | | | | | |
| | | [MOE](G)[sP].[MOE](T)[sP].[MOE] | | | | | | |
| | | (G)[sP].[MOE](G)[sP].[MOE](T)}$$$ | | | | | | |
| | | $V2.0 | | | | | | |
| 34 | GGTCAAG | RNA1{[MOE](G)[sP].[MOE](G)[sP]. | ACCACA | 39 | 449 | 466 | 44345571 | 44345588 |
| | AATGGTG | [MOE](T)[MsNP].[MOE]([5meC]) | CCATTCT | | | | | |
| | TGGT | [MsNP].[MOE](A)[MsNP].[MOE](A) | TGACC | | | | | |
| | | [MsNP].[MOE](G)[sP].[MOE](A)[sP]. | | | | | | |
| | | [MOE](A)[sP].[MOE](T)[sP].[MOE] | | | | | | |
| | | (G)[sP].[MOE](G)[sP].[MOE](T)[sP]. | | | | | | |
| | | [MOE](G)[sP].[MOE](T)[sP].[MOE] | | | | | | |
| | | (G)[sP].[MOE](G)[sP].[MOE](T)}$$$ | | | | | | |
| | | $V2.0 | | | | | | |
| 35 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[MsNP]. | CCATTCT | | | | | |
| | TGGT | [MOE](A)[MsNP].[MOE](A)[MsNP]. | TGACC | | | | | |
| | | [MOE](G)[MsNP].[MOE](A)[sP]. | | | | | | |
| | | [MOE](A)[sP].[MOE](T)[sP].[MOE] | | | | | | |
| | | (G)[sP].[MOE](G)[sP].[MOE](T)[sP]. | | | | | | |
| | | [MOE](G)[sP].[MOE](T)[sP].[MOE] | | | | | | |
| | | (G)[sP].[MOE](G)[sP].[MOE](T)}$$$ | | | | | | |
| | | $V2.0 | | | | | | |
| 36 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[MsNP].[MOE](A)[MsNP]. | TGACC | | | | | |
| | | [MOE](G)[MsNP].[MOE](A)[MsNP]. | | | | | | |
| | | [MOE](A)[sP].[MOE](T)[sP].[MOE] | | | | | | |
| | | (G)[sP].[MOE](G)[sP].[MOE](T)[sP]. | | | | | | |
| | | [MOE](G)[sP].[MOE](T)[sP].[MOE] | | | | | | |
| | | (G)[sP].[MOE](G)[sP].[MOE](T)}$$$ | | | | | | |
| | | $V2.0 | | | | | | |
| 37 | GGTCAAG | RNA1{[MOE](G)[sP].[MOE](G)[sP]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[MsNP]. | TGACC | | | | | |
| | | [MOE](G)[MsNP].[MOE](A)[MsNP]. | | | | | | |
| | | [MOE](A)[MsNP].[MOE](T)[sP].[MOE] | | | | | | |
| | | (G)[sP].[MOE](G)[sP].[MOE](T)[sP]. | | | | | | |
| | | [MOE](G)[sP].[MOE](T)[sP].[MOE] | | | | | | |
| | | (G)[sP].[MOE](G)[sP].[MOE](T)}$$$ | | | | | | |
| | | $V2.0 | | | | | | |
| 38 | GGTCAAG | RNA1{[MOE](G)[sP].[MOE](G)[P]. | ACCACA | 39 | 449 | 466 | 44345571 | 44345588 |
| | AATGGTG | [MOE](T)[sP].[MOE]([5meC])[sP]. | CCATTCT | | | | | |
| | TGGT | [MOE](A)[sP].[MOE](A)[sP].[MOE] | TGACC | | | | | |
| | | (G)[MsNP].[MOE](A)[MsNP].[MOE] | | | | | | |
| | | (A)[MsNP].[MOE](T)[MsNP].[MOE] | | | | | | |
| | | (G)[sP].[MOE](G)[sP].[MOE](T)[sP]. | | | | | | |
| | | [MOE](G)[sP].[MOE](T)[sP].[MOE] | | | | | | |
| | | (G)[sP].[MOE](G)[sP].[MOE](T)}$$$ | | | | | | |
| | | $V2.0 | | | | | | |

1. An antisense oligonucleotide, wherein the antisense oligonucleotide is 8-40 nucleotides in length and comprises a contiguous nucleotide sequence of 8-40 nucleotides in length which is complementary to a splice regulation site of the human progranulin pre-mRNA transcript, wherein the contiguous nucleotide sequence comprises one or more methanesulfonyl phosphoramidate internucleotide linkages.

2. The antisense oligonucleotide according to claim 1, wherein the human progranulin pre-mRNA transcript comprises the exon 1, intron 1, and exon 2 sequence of the human progranulin pre-mRNA transcript (SEQ ID NO: 1).

3. The antisense oligonucleotide according to claim 1, wherein the contiguous nucleotide sequence is complementary to SEQ ID NO: 39.

4. The antisense oligonucleotide according to claim 3, wherein the contiguous nucleotide sequence is SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, or SEQ ID NO: 38, or at least 8 contiguous nucleotides thereof.

5-12. (canceled)

13. The antisense oligonucleotide according to claim 1, wherein:
(i) the contiguous nucleotide sequence comprises 1, 2, 3, 4, 5, 6, 7, 8, 9, 10, 11, 12, 13, 14, 15, 16, 17, or more methanesulfonyl phosphoramidate internucleotide linkages;
(ii) the contiguous nucleotide sequence comprises one or more phosphorothioate internucleotide linkages;
(iii) at least about 75%, at least about 80%, at least about 85%, at least about 90%, at least about 95%, or about 100% of the internucleotide linkages of the contiguous nucleotide sequence are modified; and/or
(iv) all of the internucleotide linkages positioned between the nucleotides on the contiguous nucleotide sequence are modified.

14-17. (canceled)

18. The antisense oligonucleotide according to claim 1, wherein all the internucleotide linkages present in the antisense oligonucleotide are selected from phosphorothioate internucleotide linkages and methanesulfonyl phosphoramidate internucleotide linkages.

19. The antisense oligonucleotide according to claim 1, wherein the antisense oligonucleotide, or contiguous nucleotide sequence thereof, comprises one or more modified nucleosides.

20. The antisense oligonucleotide according claim 19, wherein the contiguous nucleotide sequence comprises one or more 2′-O-methoxyethyl-RNA (2′-MOE) nucleosides.

21. The antisense oligonucleotide according to claim 20, wherein:
(i) the contiguous nucleotide sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 2′-O-methoxyethyl-RNA (2′-MOE) nucleosides;
(ii) the contiguous nucleotide sequence comprises at least 10%, at least 20%, at least 30%, at least 40%, at least 50%, at least 60%, at least 70%, at least 80%, at least 90%, or 100% 2′-O-methoxyethyl-RNA (2′-MOE) nucleosides; and/or
(iii) all of the nucleosides of the contiguous nucleotide sequence are 2′-O-methoxyethyl-RNA (2′-MOE) nucleosides.

22-23. (canceled)

24. The antisense oligonucleotide according to claim 1, wherein the antisense oligonucleotide has the structure:

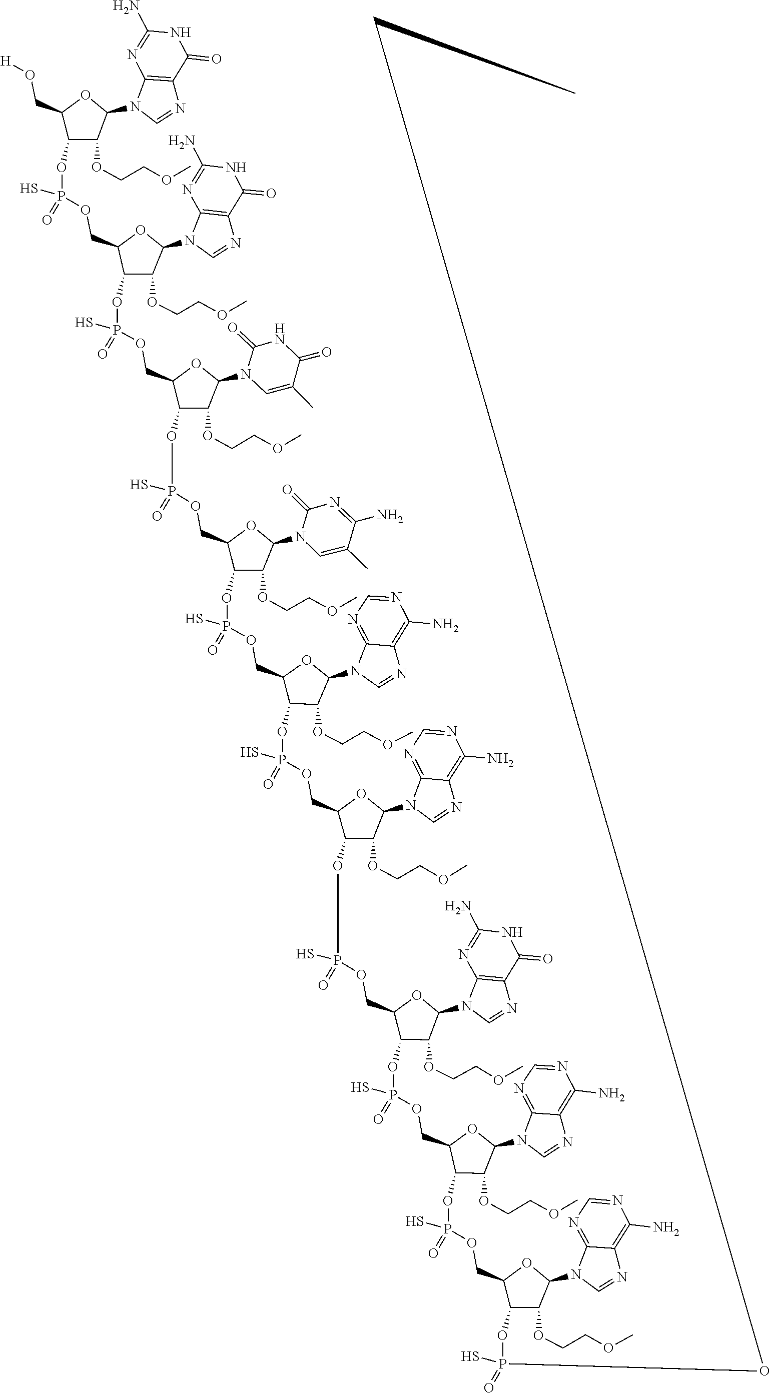

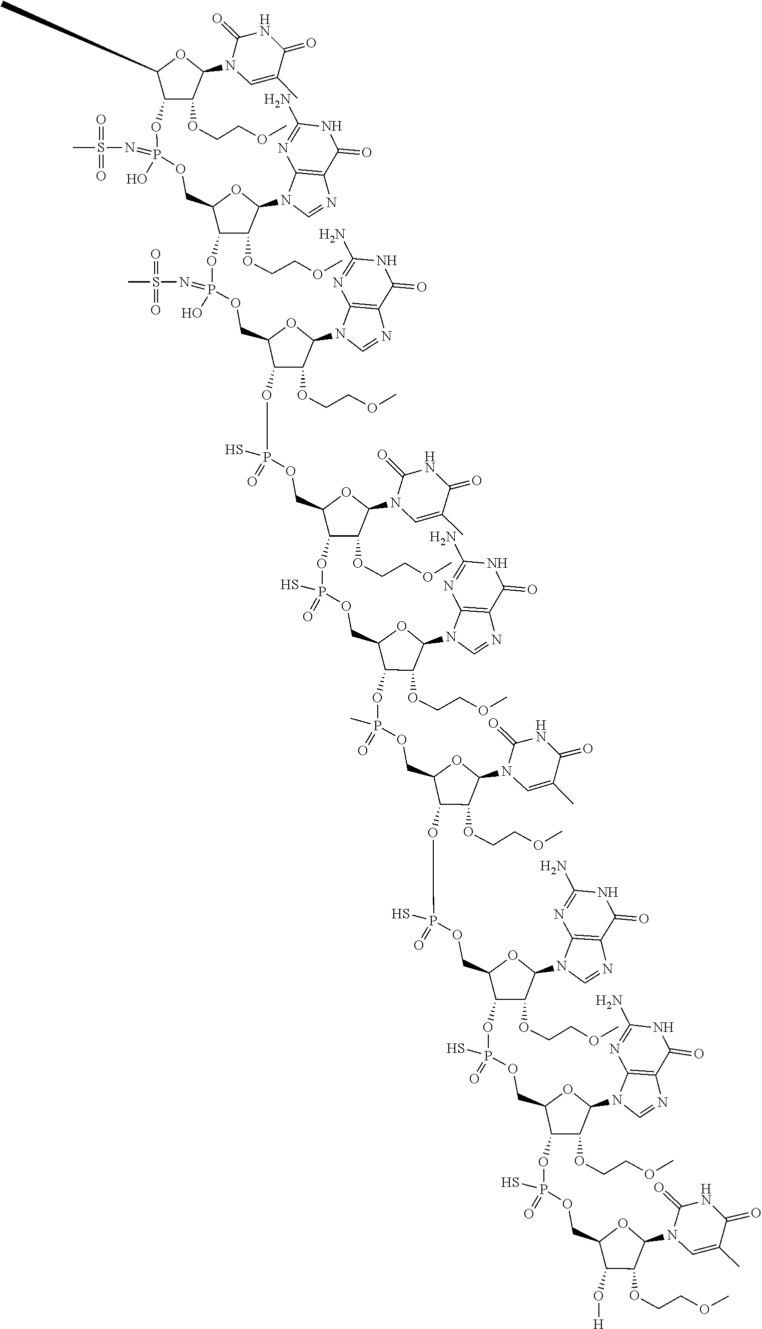

25. The antisense oligonucleotide according to claim 1, wherein the antisense oligonucleotide has the structure:

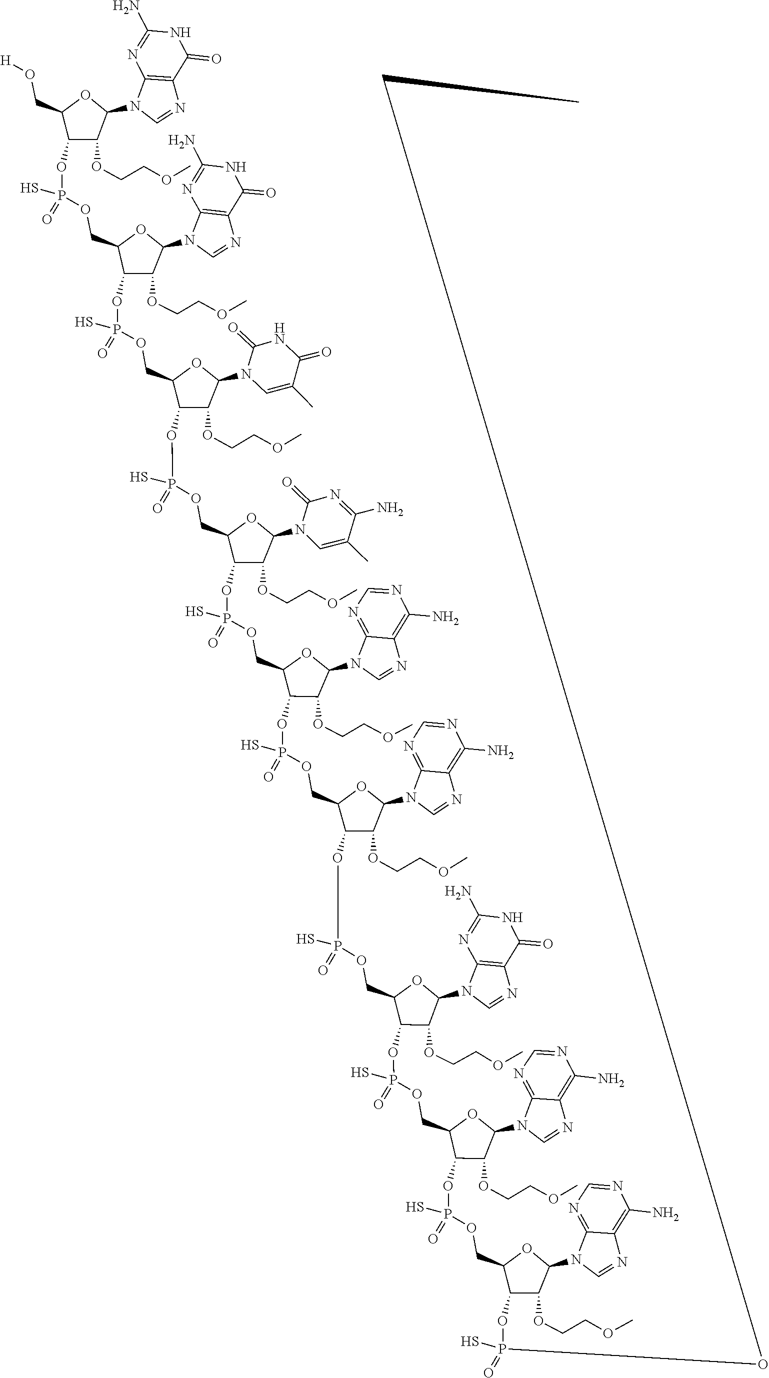

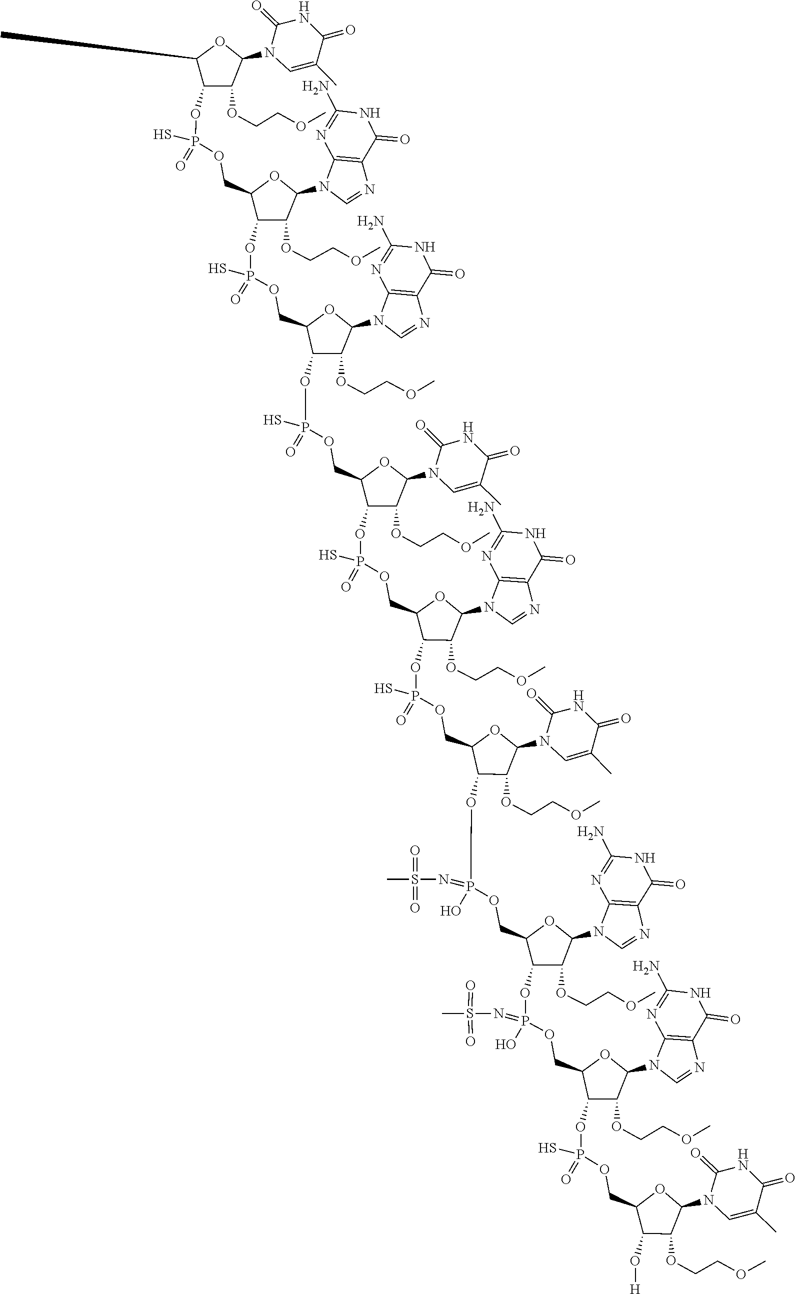

26. The antisense oligonucleotide according to claim 1, wherein the antisense oligonucleotide has the structure:

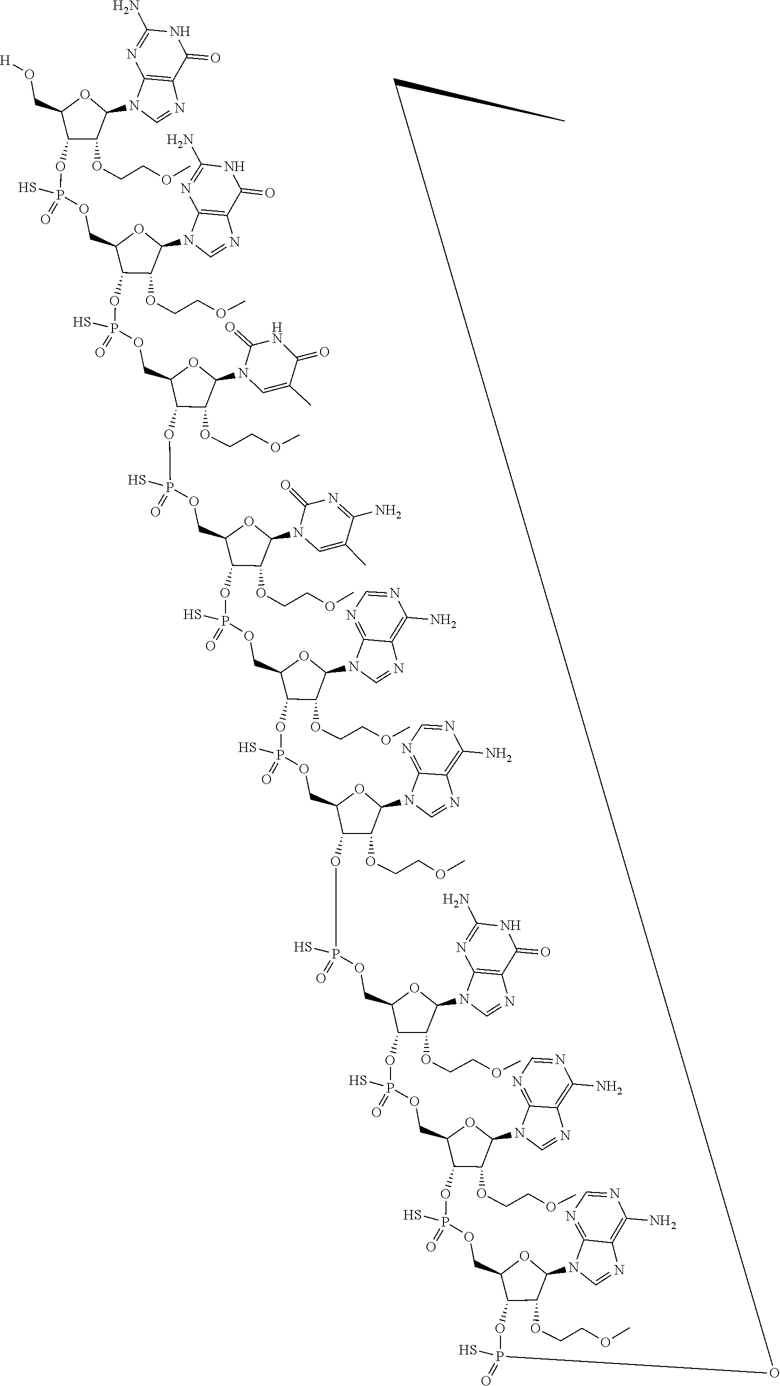

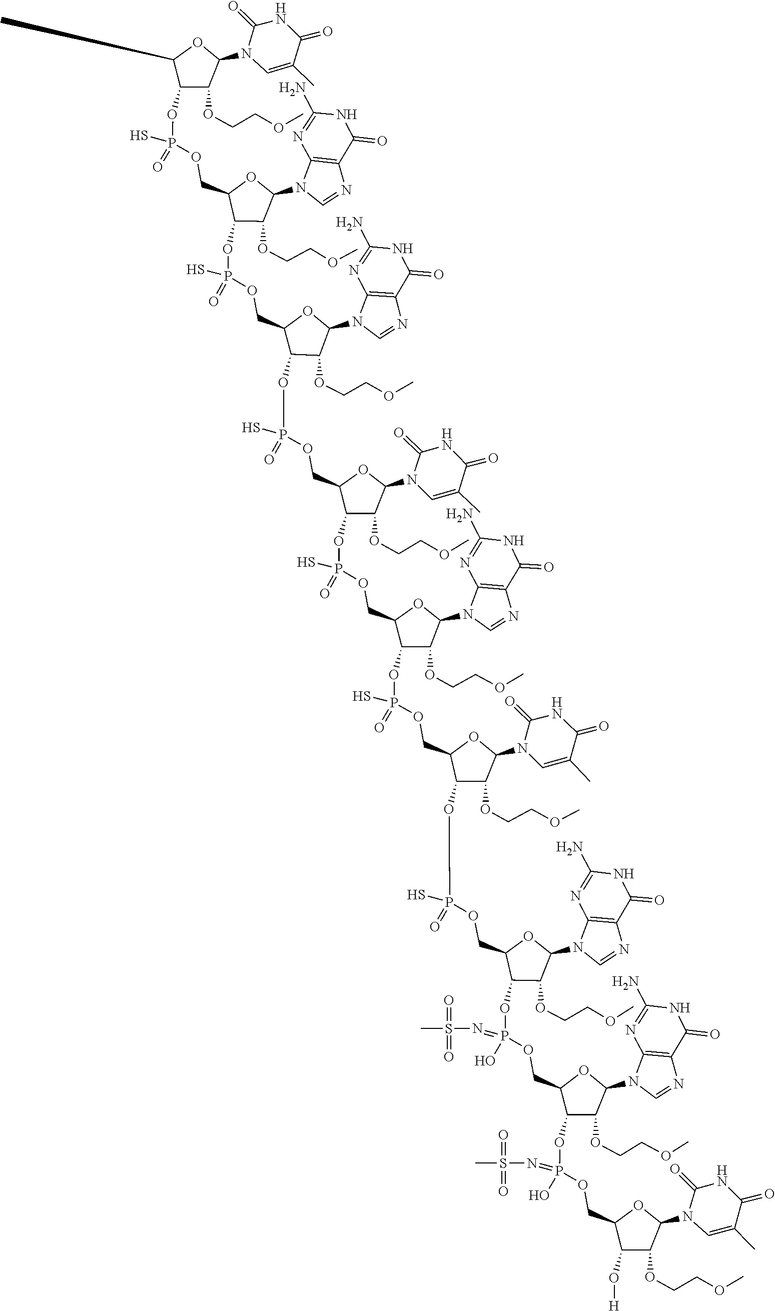

27. The antisense oligonucleotide according to claim 1, wherein the antisense oligonucleotide has the structure:

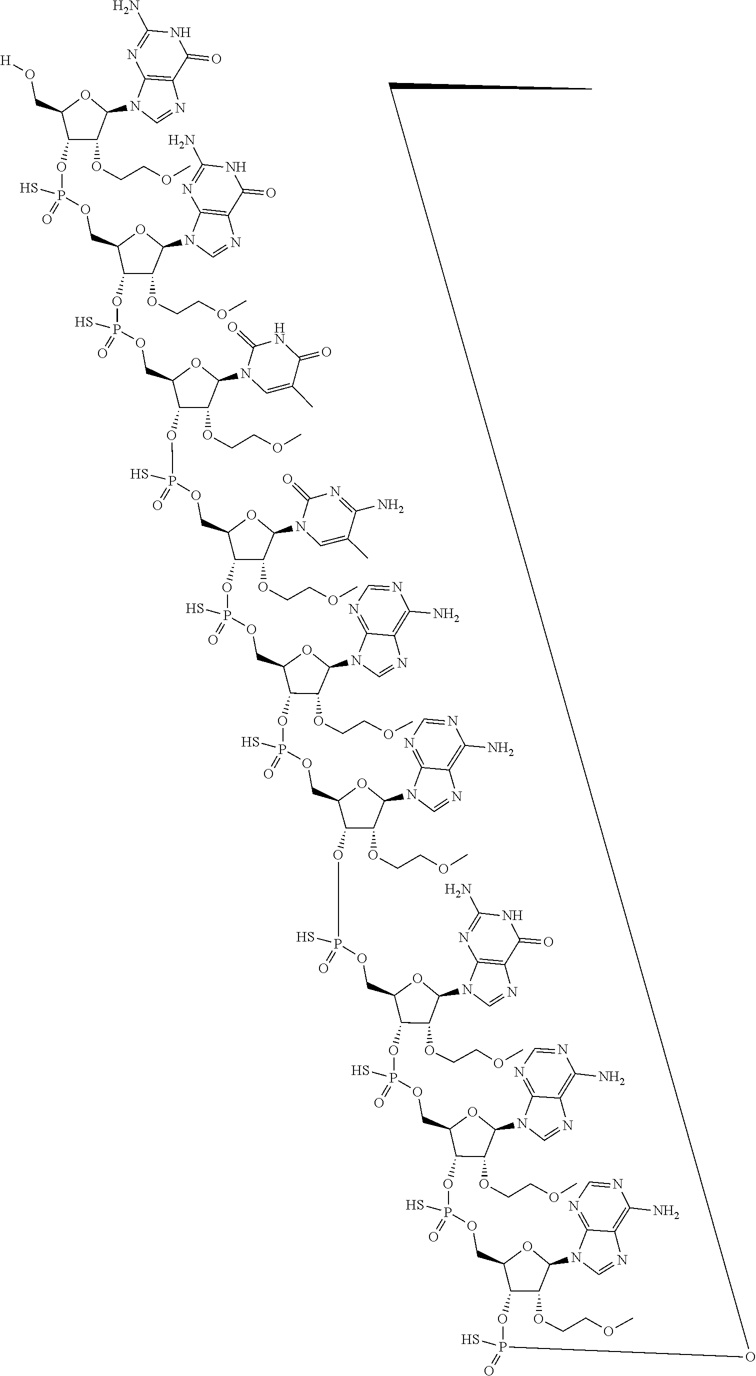

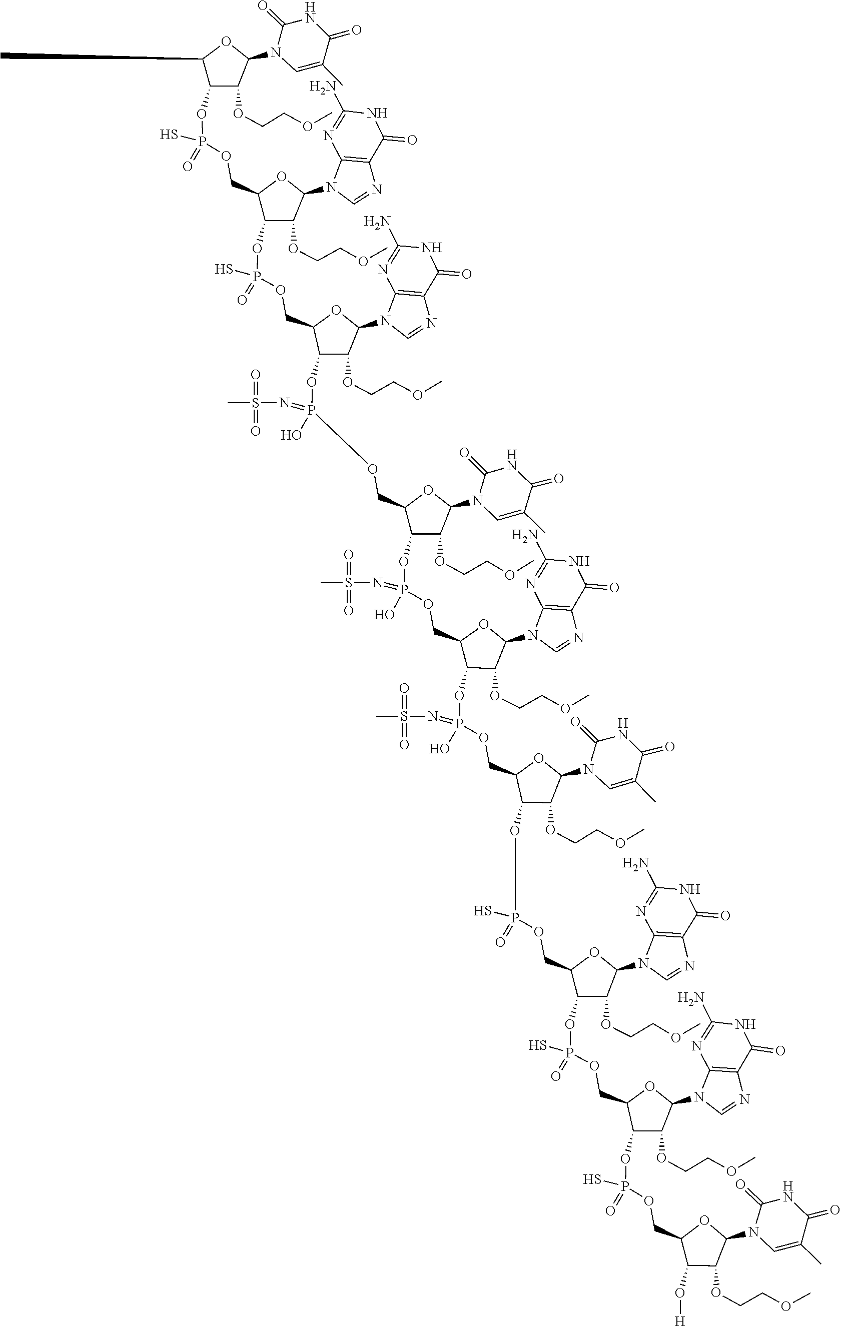

28. The antisense oligonucleotide according to claim 1, wherein the antisense oligonucleotide has the structure:

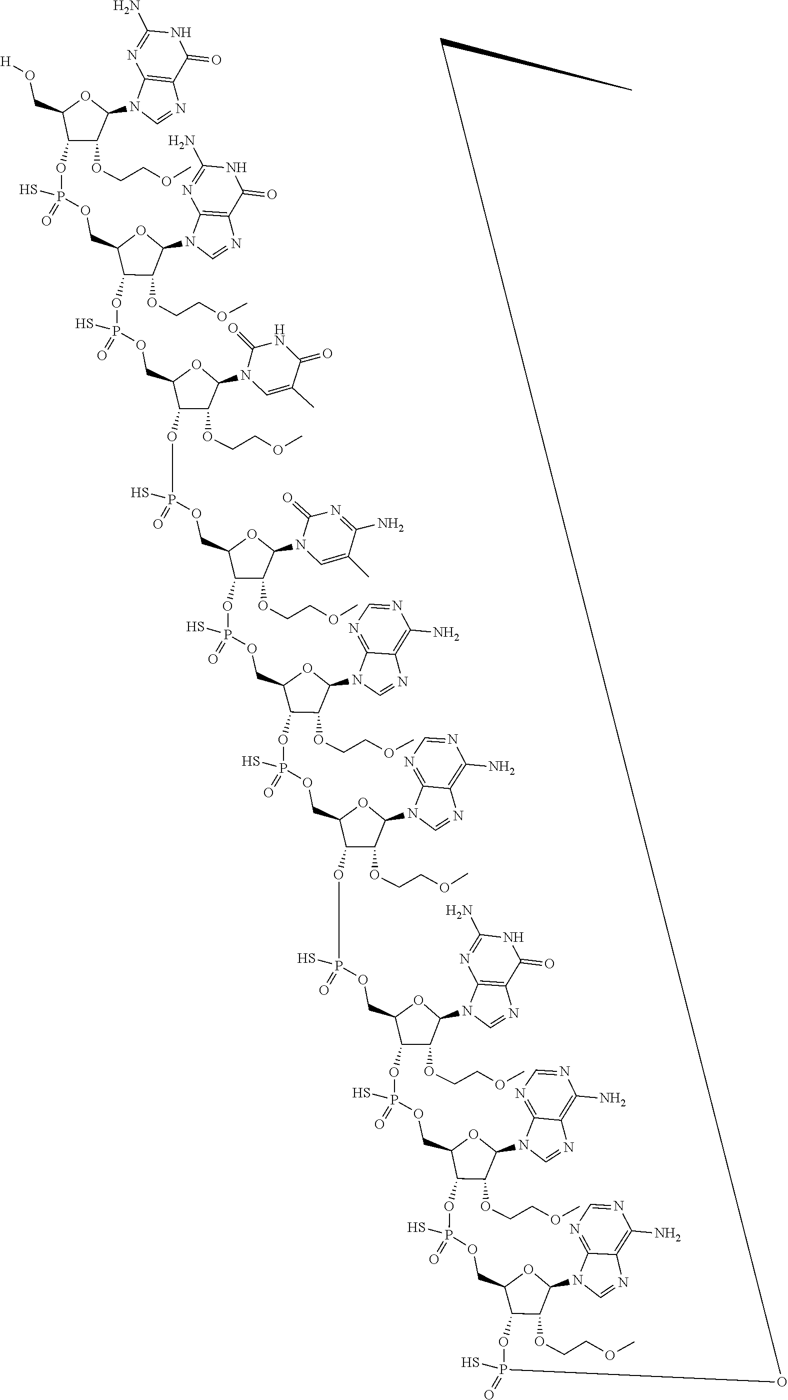

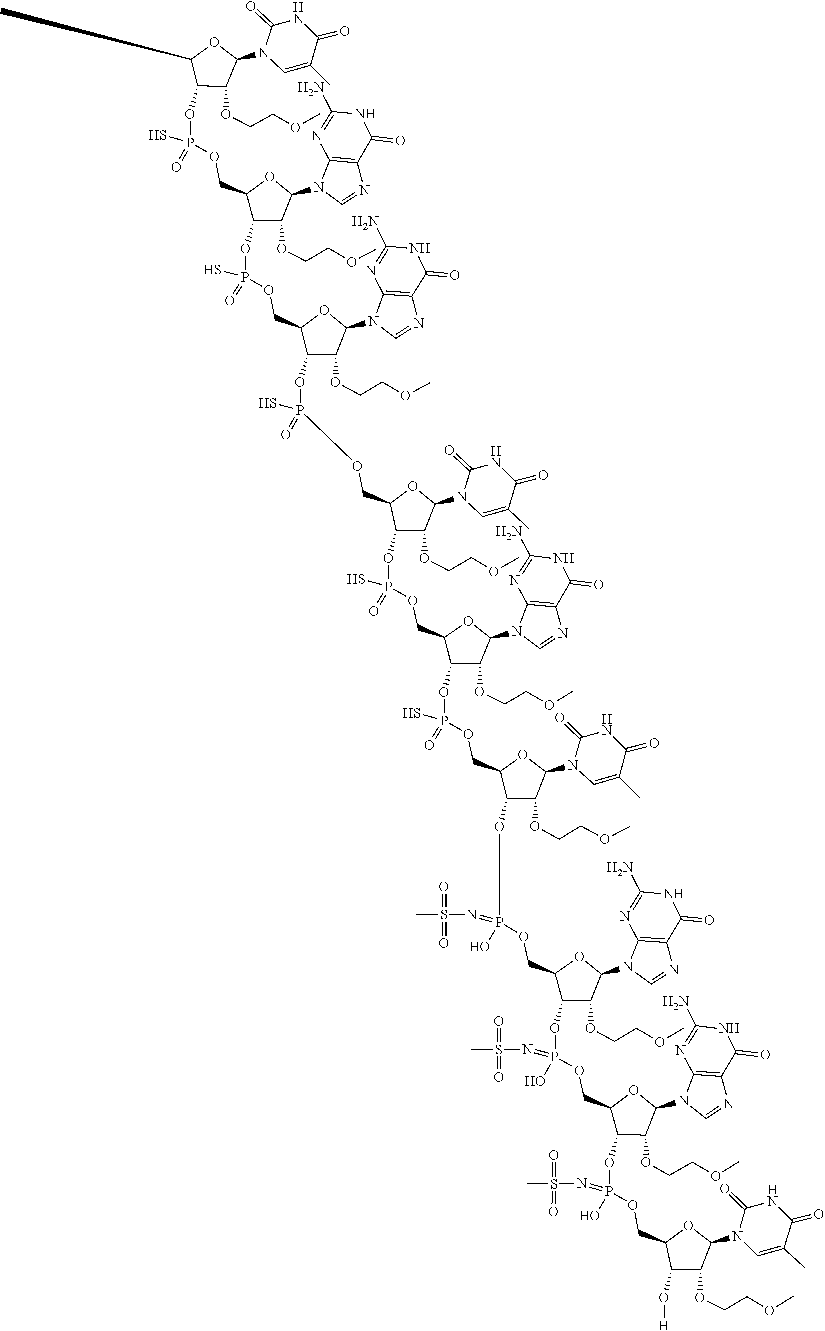

29. The antisense oligonucleotide according to claim 1, wherein the antisense oligonucleotide has the structure:

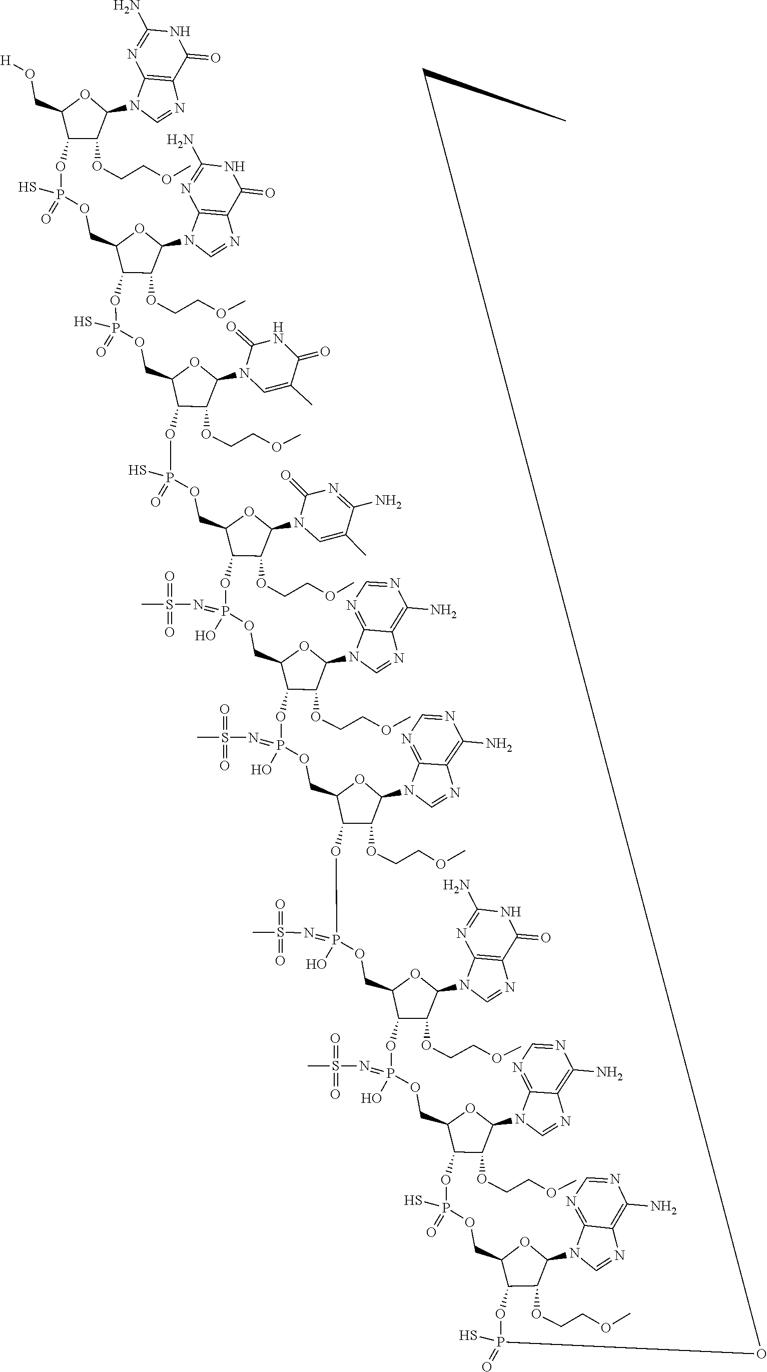

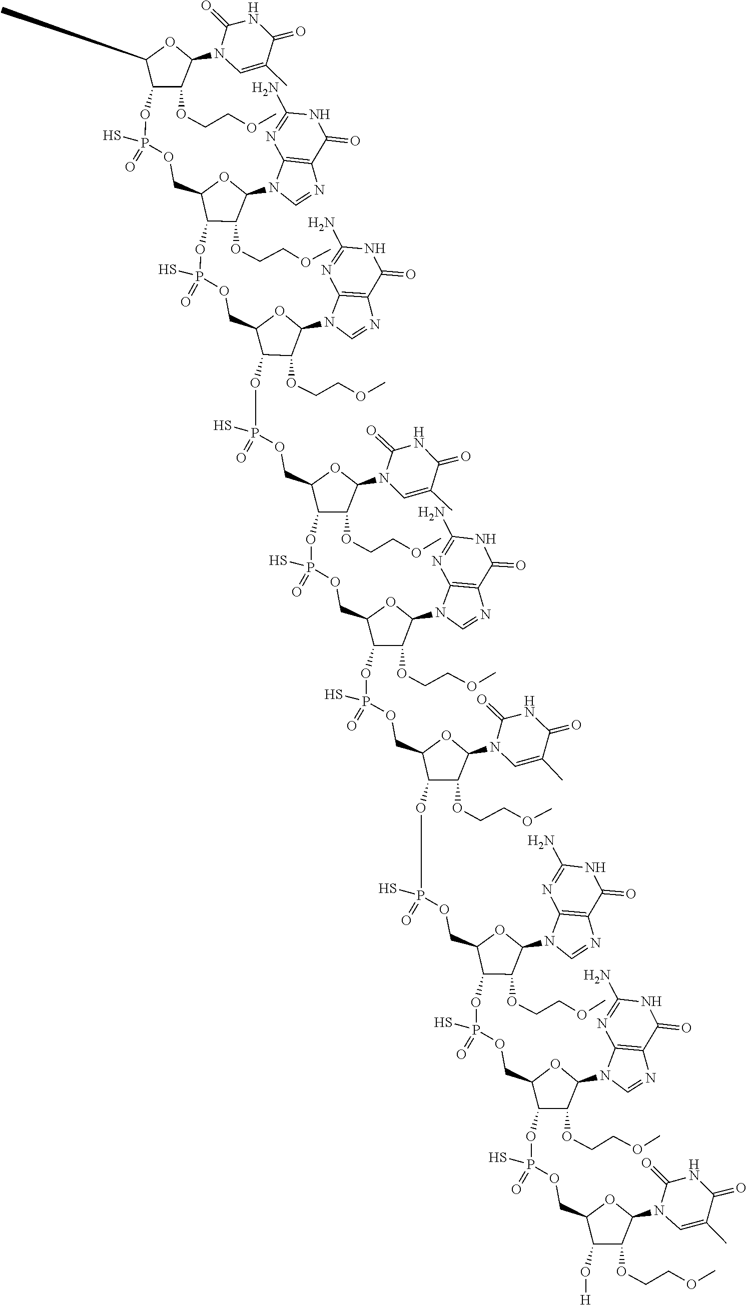

30. The antisense oligonucleotide according to claim 1, wherein the oligonucleotide is an oligonucleotide compound GGTCAAGAATGGTGTGGT (SEQ ID NO: 11, SEQ ID NO: 15, SEQ ID NO: 16, SEQ ID NO: 28, SEQ ID NO: 31, or SEQ ID NO: 35), wherein all the nucleosides are 2′-O-methoxyethyl-RNA (2′-MOE) nucleosides, the C is 5-methyl cytosine and all internucleoside linkages are phosphorothioate internucleoside linkages, methanesulfonyl phosphoramidate internucleoside linkages, or a combination thereof.

31. A pharmaceutical composition comprising the antisense oligonucleotide according to claim 1, and a pharmaceutically acceptable diluent, solvent, carrier, salt, and/or adjuvant.

32. A method of treating a neurological disease in a subject, comprising administering to the subject the antisense oligonucleotide according to claim 1, or a pharmaceutical composition thereof comprising the antisense oligonucleotide.

33. A method of treating a progranulin haploinsufficiency or a related disorder in a subject, comprising administering to the subject the antisense oligonucleotide according to claim 1, or a pharmaceutical composition thereof comprising the antisense oligonucleotide.

34. An in vivo or in vitro method for enhancing the expression of the Exon1-Exon2 progranulin splice variant in a cell which is expressing progranulin, said method comprising administering an antisense oligonucleotide according to claim 1, or a pharmaceutical composition thereof comprising the antisense oligonucleotide.
